(19) United States
(12) Patent Application Publication
(10) Pub. No.: US 2024/0068007 A1
(43) Pub. Date: Feb. 29, 2024

(54) BIOMARKERS OF RISK FOR INFECTION

(71) Applicant: University of Chicago, Chicago, IL (US)

(72) Inventors: Eric Gerd Pamer, Chicago, IL (US); Nicholas Dylla, Chicago, IL (US); Christopher Lehmann, Chicago, IL (US)

(21) Appl. No.: 18/236,022

(22) Filed: Aug. 21, 2023

Related U.S. Application Data
(60) Provisional application No. 63/458,986, filed on Apr. 13, 2023.
(60) Provisional application No. 63/373,811, filed on Aug. 29, 2022.

(51) Int. Cl. C12Q 1/10; G01N 33/92

(52) U.S. Cl. CPC C12Q 1/10; G01N 33/92; G01N 2800/50

(57) ABSTRACT

Provided herein are biomarkers associated with risk of bacterial infection, particularly hospital inquired infections, such as following surgeries. In particular, provided herein are fecal biomarkers (e.g., microbiomic, metabolomic, etc.) that correlate with elevated risk of infection in subjects following surgery (e.g., organ transplant surgery), and methods of treating and/or preventing such infections.

### PRIORITY STATEMENT

This application claims priority to U.S. Provisional Patent Application No. 63/373,811, filed Aug. 29, 2022 and U.S. Provisional Patent application No. 63/458,986, filed Apr. 13, 2023, the entire contents of each of which are incorporated herein by reference.

### FIELD

Provided herein are biomarkers associated with risk of bacterial infection, particularly hospital acquired infections, such as following surgeries. In particular, provided herein are fecal biomarkers (e.g., microbiomic and metabolomic biomarkers.) that indicate elevated risk of infection in subjects following surgery (e.g., organ transplant surgery), and methods of treating and/or preventing such infections.

### BACKGROUND

Liver transplantation is the definitive treatment for many acute, chronic, and malignant liver diseases. Despite advances is prophylaxis, patient selection, donor screening, and surgical techniques, infection remains a significant cause of morbidity and mortality in liver transplant recipients. Enterococcus species and members of the order Enterobacterales, such as Escherichia coli and Klebsiella pneumonia, remain among the most common causes of infection in the post-transplant period. The clinical impact of these infections is compounded by rising rates of antibiotic resistance, with vancomycin resistant Enterococcus faecium (VRE) and multiple drug resistant Enterobacterales making outsized contributions to morbidity and mortality. Accordingly, what is needed are methods for preventing and/or treating infection following surgery.

### SUMMARY

In some aspects, provided herein are methods of assessing risk of infection in a subject. In some embodiments, provided herein is a method of assessing the risk of infection for a subject comprising assessing the level of one or more biomarkers of infection in the gut of the subject. In some embodiments, the subject is hospitalized. In some embodiments, the subject is preparing to undergo a surgery or has received a surgery. In some embodiments, the surgery is an organ transplant. For example, in some embodiments the surgery is an organ transplant such as a liver transplant, a kidney transplant, an intestinal transplant, a heart transplant, or a lung transplant. In some embodiments, the organ transplant is a liver transplant.

In some embodiments, the level of the one or more biomarkers are assessed in a fecal sample from the subject. In some embodiments, the fecal sample is obtained from the subject within 30 days of a surgery. For example, in some embodiments the fecal sample is obtained from the subject 30 days or less prior to surgery (e.g. prior to a transplant). As another example, in some embodiments the fecal sample is obtained from the subject no more than 30 days after surgery (e.g. after transplant). For example, in some embodiments the fecal sample is obtained 30 days or less, 25 days or less, 20 days or less, 15 days or less, or 5 days or less prior to a surgery. As another example, in some embodiments the fecal sample is obtained 30 days or less, 25 days or less, 20 days or less, 15 days or less, 10 days or less, or 5 days or less after surgery.

In some embodiments, the one or more biomarkers are selected from the microbiomic and metabolomic biomarkers herein. In some embodiments, the one or more biomarkers of infection comprise one or more microbiomic biomarkers. In some embodiments, the more microbiomic biomarkers comprise a relative abundance of Enterococcus species and/or a relative abundance of Enterobacterales species in a fecal sample obtained from the subject. In some embodiments, the subject is indicated to be at risk of having or currently experiencing an infection when the relative abundance of Enterococcus species in the fecal sample is greater than or equal to a threshold value for Enterococcus species; and/or the relative abundance of Enterobacterales species in the fecal sample is greater than or equal to a threshold value for Enterobacterales species. In some embodiments, the threshold value for Enterococcus species is 19.9% and/or the threshold value for Enterobacterales species is 2.5%.

In some embodiments, the one or more biomarkers of infection comprise one or more metabolomic biomarkers. For example, in some embodiments the one or more metabolomic biomarkers are selected from tyramine, kynurenine, acetate, butyrate, propionate, taurocholic acid, cholic acid, 3-oxolithocholic acid, chenodeoxycholic acid, 12-oxochenodeoxycholic acid, deoxycholic acid, isodeoxycholic acid, and lithocholic acid. In some embodiments, increased levels of one or more of tyramine, kynurenine, taurocholic acid, cholic acid, chenodeoxycholic acid, or 12-oxochenodeoxycholic acid and/or decreased expression of one or more of acetate, butyrate, propionate, 3-oxolithocholic acid, deoxycholic acid, isodeoxycholic acid, lithocolic acid, tyrosine, valerate, hexanotate, indole-3-propionate, allolithocholic acid, isolithocholic acid, alloisolithocholic acid, crotonate, isobutyrate, or isovalerate in a fecal sample obtained from the subject indicates that the subject is at risk of having or currently experiencing an infection. In some embodiments, increased levels of two or more of tyramine, kynurenine, taurocholic acid, cholic acid, chenodeoxycholic acid, or 12-oxochenodeoxycholic acid and/or decreased expression of two or more of acetate, butyrate, propionate, 3-oxolithocholic acid, deoxycholic acid, isodeoxycholic acid, lithocolic acid, tyrosine, valerate, hexanotate, indole-3-propionate, allolithocholic acid, isolithocholic acid, alloisolithocholic acid, crotonate, isobutyrate, or isovalerate in a fecal sample obtained from the subject indicates that the subject is at risk of having or currently experiencing an infection. In some embodiments, increased levels of three or more of tyramine, kynurenine, taurocholic acid, cholic acid, chenodeoxycholic acid, or 12-oxochenodeoxycholic acid and/or decreased expression of three or more, four or more, five or more, six or more, seven or more, eight or more, nine or more, or at least ten of acetate, butyrate, propionate, 3-oxolithocholic acid, deoxycholic acid, isodeoxycholic acid, lithocolic acid, tyrosine, valerate, hexanotate, indole-3-propionate, allolithocholic acid, isolithocholic acid, alloisolithocholic acid, crotonate, isobutyrate, or isovalerate in a fecal sample obtained from the subject indicates that the subject is at risk of having or currently experiencing an infection.

In some aspects, provided herein are methods of treating a subject. In some embodiments, the methods comprise assessing risk of infection as described herein, and administering an appropriate therapy to the subject when the risk of infection is determined to be increased. In some embodiments, the method of treating a subject comprises assessing the subject's risk of infection by the methods described herein, and providing microbiome augmentation to the subject when the subject is determined to be at increased risk of infection. In some embodiments, the methods not performing microbiome augmentation when the subject is determined to be at decreased risk of infection. In some embodiments, microbiome augmentation comprises fecal augmentation therapy. In some embodiments, microbiome augmentation comprises treatment with one or more antibiotics. In some embodiments, microbiome augmentation comprises treatment with one or more probiotics. In some embodiments, a combination of microbiome augmentation therapies (e.g. one or more of fecal augmentation therapy, antibiotic treatment, and/or probiotic treatment) is provided to the subject.

In some aspects, provided herein is a method comprising assessing the level of one or more microbiomic biomarkers and/or one or more metabolomic biomarkers in a fecal sample obtained from a subject within 30 days of a surgery on the subject. For example, in some embodiments the fecal sample is obtained 30 days or less, 25 days or less, 20 days or less, 15 days or less, or 5 days or less prior to a surgery. As another example, in some embodiments the fecal sample is obtained 30 days or less, 25 days or less, 20 days or less, 15 days or less, 10 days or less, or 5 days or less after surgery. In some embodiments, the one or more microbiomic biomarkers comprise a relative abundance of Enterococcus species and/or a relative abundance of Enterobacterales species in the fecal sample. In some embodiments, the subject is indicated to be at risk of having or currently experiencing an infection when the relative abundance of Enterococcus species in the fecal sample is greater than or equal to a threshold value for Enterococcus species; and/or the relative abundance of Enterobacterales species in the fecal sample is greater than or equal to a threshold value for Enterobacterales species. In some embodiments, the threshold value for Enterococcus species is 19.9% and/or the threshold value for Enterobacterales species is 2.5%. In some embodiments, the one or more metabolomic biomarkers are selected from tyramine, kynurenine, acetate, butyrate, propionate, taurocholic acid, cholic acid, 3-oxolithocholic acid, chenodeoxycholic acid, 12-oxochenodeoxycholic acid, deoxycholic acid, isodeoxycholic acid, and lithocholic acid.

In some embodiments, increased levels of one or more of tyramine, kynurenine, taurocholic acid, cholic acid, chenodeoxycholic acid, or 12-oxochenodeoxycholic acid and/or decreased expression of one or more of acetate, butyrate, propionate, 3-oxolithocholic acid, deoxycholic acid, isodeoxycholic acid, lithocolic acid, tyrosine, valerate, hexanotate, indole-3-propionate, allolithocholic acid, isolithocholic acid, alloisolithocholic acid, crotonate, isobutyrate, and/or isovalerate in the fecal sample indicates that the subject is at risk of having or currently experiencing an infection. In some embodiments, increased levels of two or more of tyramine, kynurenine, taurocholic acid, cholic acid, chenodeoxycholic acid, or 12-oxochenodeoxycholic acid and/or decreased expression of two or more of acetate, butyrate, propionate, 3-oxolithocholic acid, deoxycholic acid, isodeoxycholic acid, lithocolic acid, tyrosine, valerate, hexanotate, indole-3-propionate, allolithocholic acid, isolithocholic acid, alloisolithocholic acid, crotonate, isobutyrate, or isovalerate in a fecal sample obtained from the subject indicates that the subject is at risk of having or currently experiencing an infection. In some embodiments, increased levels of three or more of tyramine, kynurenine, taurocholic acid, cholic acid, chenodeoxycholic acid, or 12-oxochenodeoxycholic acid and/or decreased expression of three or more, four or more, five or more, six or more, seven or more, eight or more, nine or more, or at least ten of acetate, butyrate, propionate, 3-oxolithocholic acid, deoxycholic acid, isodeoxycholic acid, lithocolic acid, tyrosine, valerate, hexanotate, indole-3-propionate, allolithocholic acid, isolithocholic acid, alloisolithocholic acid, crotonate, isobutyrate, or isovalerate in a fecal sample obtained from the subject indicates that the subject is at risk of having or currently experiencing an infection.

In some embodiments, the method further comprises providing microbiome augmentation to the subject when the subject is determined to be at risk of having or currently experiencing an infection. In some embodiments, the surgery is an organ transplant. For example, in some embodiments the surgery is an organ transplant such as a liver transplant, a kidney transplant, an intestinal transplant, a heart transplant, or a lung transplant. In some embodiments, the organ transplant is a liver transplant.

### BRIEF DESCRIPTION OF THE DRAWINGS

FIGS. 1A-1J show microbiome compositions of liver transplant (LT) recipients vary widely. (FIG. 1A) Fecal microbiome composition plots of LT patients and healthy donors (HD) vertically organized by relative abundance and color coded by taxa. Individual samples were ordered horizontally by Shannon diversity. (FIG. 1B) taxUMAP1 plot of taxonomic composition, HD are denoted as triangles. Samples are color coded by the most abundant taxon and size determined by the relative abundance of that taxon. (FIGS. 1C-1E) Comparison of Alpha diversity between LT diversity groups and between high diversity and HD using (FIG. 1C) Inverse Simpson, (FIG. 1D) Shannon, and (FIG. 1E) Richness. (FIGS. 1F-1J) Comparison of relative abundance of select taxa between LT diversity groups and HD. (FIG. 1F) Lachnospiraceae; (FIG. 1G) Enterococcus; (FIG. 1H) Enterobacterales; (FIG. 1I) Bacteroidetes; (FIG. 1J) Oscilospiraceae. Significance tested by Kruskal-Wallis test *p≤0.05 **p≤0.01 ***p≤0.001 ****p≤0.0001

FIGS. 2A-2B show qualitatively measured microbiome derived fecal metabolites vary widely among LT recipients. Individual metabolite abundances represented on a colorimetric heat map by log₂ fold change from the mean between samples. The scale provided in FIG. 2A also applies to FIG. 2B, with red indicating increased abundance; blue indicating reduced abundance. Significance was measured between LT groups using the Kurskal-Wallis test and denoted on a colorimetric scale where green represents lower p-values, adjusted for multiple comparisons. HD were included for visual comparison. Add. Compounds include kynurenine pathway and phenolic aromatics. Abbreviations: 1°-Primary; 2°-Secondary; Add.-Additional; 3-Oxo-D-Or-3-Oxocheno.-1-Oxo-Deoxycholic or 3-Oxochenodeoxycholic acid, which could not be completely discriminated chromatographically and are included together.

FIGS. 3A-3I show quantitatively measured microbiome derived fecal metabolites vary widely among LT recipients. Absolute abundances of acetate, propionate, butyrate, cholic acid, glycocholic acid, taurocholic acid, deoxycholic acid, lithocholic acid, and alloisolithocholic acid compared between LT diversity groups and between high diversity LT and HD. *p≤0.05 **p≤0.01 ***p≤0.001 ****p≤0.0001

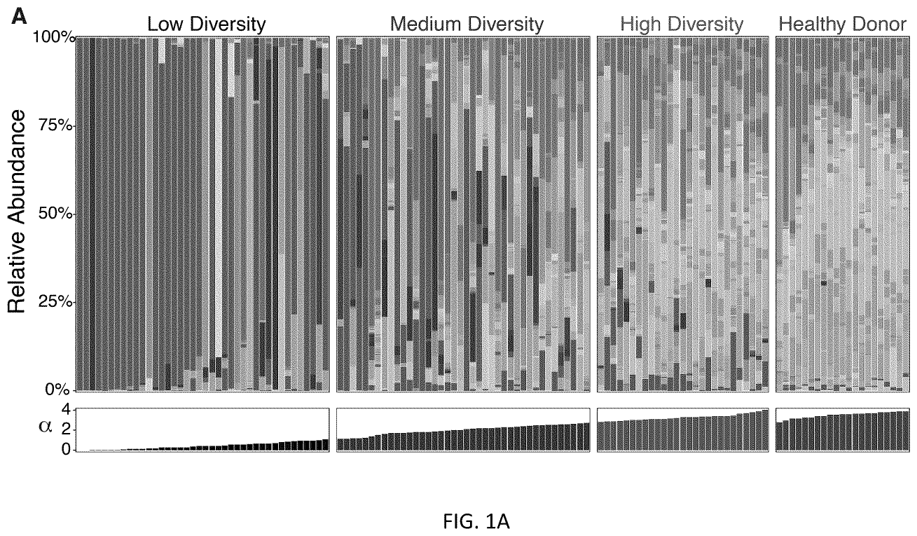

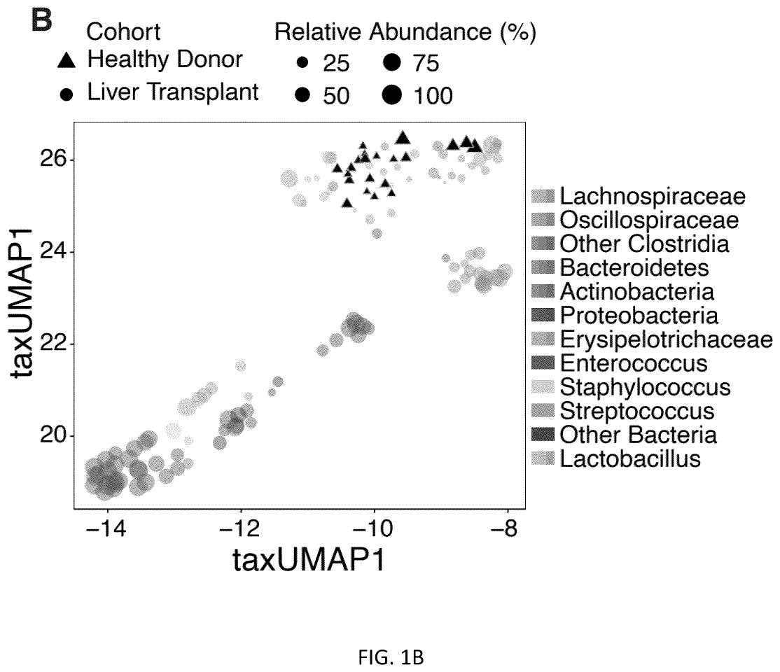

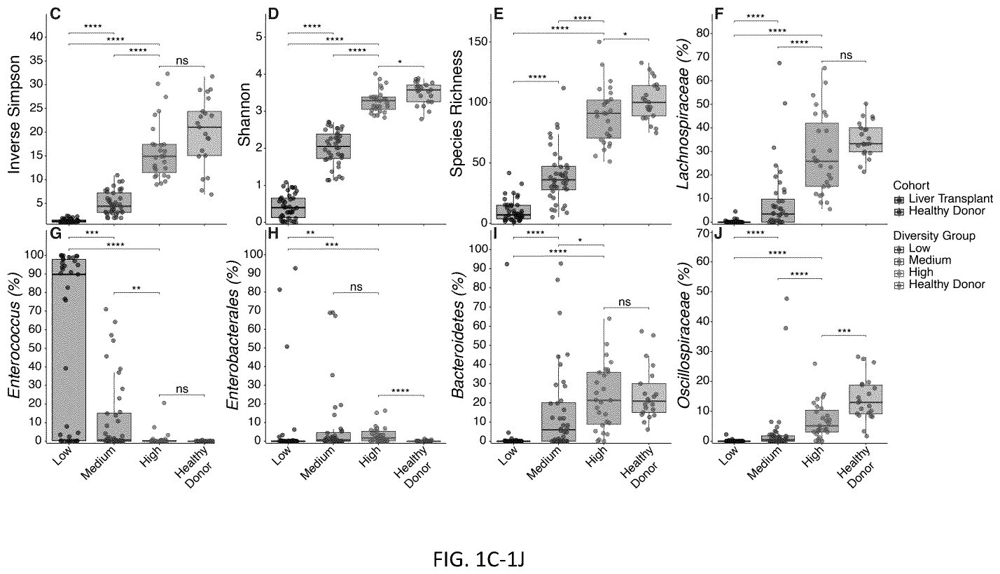

FIGS. A-4D show Enterococcus and Enterobacterales expansion in the gut microbiome predicts postoperative infection. (FIG. 4A) Fecal microbiome composition plots color coded by taxon. Plots are categorized by presence of bacterial infection and ordered by descending relative abundance of Enterococcus. Colored tiles indicate an infection caused by the denoted organism associated with that stool sample. For taxonomic color palate refer to FIG. 1. (FIG. 4B) Receiver operator curve using Enterococcus abundance to predict Enterococcus infection. Cut point determined by Youden Index to optimize both sensitivity and specificity. 95% Confidence intervals for Accuracy (0.74-0.91), Specificity (59-94%), and Sensitivity (65-100%). (FIG. 4C) Fecal microbiome composition plots organized by relative abundance of Enterobacterales. (FIG. 4D) Receiver operator curve using Enterobacterales abundance to predict Enterobacterales infection. 95% Confidence intervals Accuracy (0.82-0.96), Specificity (72-94%), and Sensitivity (100-100%). Abbreviations: AUC-Area under the curve; ACC-Accuracy; Spec.-Specificity; Sens.-Sensitivity.

FIGS. 5A-5K show microbiome derived fecal metabolites are enriched in LT recipients with and without Enterococcus expansion. (FIG. 5A) Volcano plot comparing Log₂ fold change of qualitative metabolite concentration from the mean, and Logic, scale p-value adjusted for multiple comparisons. Only metabolites with Log₂ fold change ≥+/−1 and p≤0.05 were labeled. (B-K) Quantitative metabolite comparison of select metabolites. (FIG. 5B) Acetate (FIG. 5C) Butyrate (FIG. 5D) Propionate (FIG. 5E) Taurocholic acid (FIG. 5F) Cholic acid (FIG. 5G) 3-Oxolithocholic acid (FIG. 5H) Alloisolithocholic acid (FIG. 5I) Deoxycholic acid (FIG. 5J) Isodeoxycholic acid (FIG. 5K) Lithocholic acid. Significance tested by Kruskal-Wallis test.

FIGS. 6A-6B show microbiome derived fecal metabolites identify low, medium, and high diversity samples and post operative infection. (FIG. 6A) sPLS-DA using input matrix of sample metabolites and predicted microbial diversity group. Comparison between predicted groups was visualized on a grid with dividing lines and optimized by maximum distance between groups. Accuracy was: Low Diversity 77%, Medium Diversity 72%, and High Diversity 78%. Sensitivity ranged from 60-84%. Specificity ranged from 62-95%. (FIG. 6B) sPLS-DA using input matrix of sample metabolites and predicted postoperative infection. Comparison between outcomes was visualized on a grid with a dividing line and optimized by maximum distance between groups. Accuracy was 82.2% [73.9-89.1%], sensitivity was 63% [42.4-80.6], specificity was 88.9% [80-94.8%], and odds ratio was 13.6 [4.8-38.6].

FIG. 7 is a flow diagram of inclusion and exclusion criteria. 28 patients were excluded because they had not yet undergone a LT. 23 patients were excluded because a sample wasn't collected within −7 to 30 days post LT. 107 patients were included for analysis, 25 developed postoperative bacterial infection, 82 did not.

FIGS. 8A-8B show time to first stool collection relative to the day of liver transplant. (FIG. 8A) Histogram of first stool collections normalized to the day of transplant. The median being post operative day 4. The majority falling between day −7 and +7. (FIG. 8B) Median and IQR of day to first stool sample collected by diversity group. No significant difference existed between groups, suggesting perioperative antibiotics don't explain the difference in microbiome composition between groups.

FIGS. 9A-9B show microbiome derived fecal metabolites identify low, medium, and high diversity samples. Metabolite component loadings of sPLS-DA model (FIG. 6A). x-axis indicates magnitude of impact on each component. Each component is color coded for which outcome it correlates, Low, Medium, and High diversity. 3-Oxodeoxycholic acid and 3-Oxochenodeoxycholic acid could not be discredited chromatographically, so are included together. FIG. 9A shows metabolic components for low, medium, and high diversity group. FIG. 9B show results for the medium diversity group only.

FIGS. 10A-10B show microbiome derived fecal metabolites identify postoperative infection. Metabolite component loadings of first two components of sPSL-DA model predicting postoperative infection. (FIG. 6B) The x-axis corresponds to the magnitude of impact each metabolite has on the component. Each metabolite is color coded for infection or no infection. 3-Oxodeoxycholic acid and 3-Oxochenodeoxycholic acid could not be discredited chromatographically, and as such are included together.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a metabolite” is a reference to one or more metabolites and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “microbiome” refers to the microbes (e.g., bacteria, fungi, protists) their genetic elements (genomes) in a defined environment. The term “gut microbiome” refers to the microbes present in the digestive tract of a subject. A “microbiomic analysis” refers to the use of any suitable techniques to identify the presence/absence/level of one or more (e.g., all) microbes within a sample or environment. The term “microbiomic biomarker” refers to a species or group of microbes, the presence/absence/level of which in a sample from a subject is indicative/prognostic/diagnostic of a condition/outcome in the subject. For example, in some embodiments a microbiomic biomarker is a microbe indicative of risk of infection or current infection in a subject.

As used herein, the term “metabolome” refers to the complete set of small-molecule metabolites (e.g., metabolic intermediates, hormones signaling molecules, secondary metabolites, etc.) found within a defined biological environment or sample. The term “gut metabolome” refers to the metabolites present in the digestive tract of a subject. A “metabolomic analysis” refers to the use of any suitable techniques to identify the presence/absence/level of one or more (e.g., all) of the metabolites within a sample or environment. The term “metabolomic biomarker” refers to a small molecule metabolite, the presence/absence/level of which in a sample from a subject is indicative/prognostic/diagnostic of a condition/outcome in the subject. For example, in some embodiments a metabolomic biomarker is a small molecule metabolite indicative or risk of infection or current infection in a subject.

As used herein, the term “subject” and “patient” are used interchangeably herein and broadly refer to any animal, including but not limited to, human and non-human animals (e.g., dogs, cats, cows, horses, sheep, poultry, fish, crustaceans, etc.).

As used herein, the terms “administration” and “administering” refer to the act of giving a drug, prodrug, or other agent, or therapeutic treatment to a subject or in vivo, in vitro, or ex vivo cells, tissues, and organs. Exemplary routes of administration to the human body can be by parenteral administration (e.g., intravenously, subcutaneously, etc.).

As used herein, the term “effective amount” refers to the amount of a composition sufficient to effect beneficial or desired results. An effective amount can be administered in one or more administrations, applications or dosages and is not intended to be limited to a particular formulation or administration route.

As used herein, the terms “co-administration” and “co-administering” refer to the administration of at least two agent(s) or therapies to a subject. In some embodiments, the co-administration of two or more agents or therapies is concurrent (e.g., in a single formulation/composition or in separate formulations/compositions). In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “pharmaceutical composition” refers to the combination of an active agent with a carrier, inert or active, making the composition especially suitable for diagnostic or therapeutic use in vitro, in vivo or ex vivo.

As used herein, the term “prognosis” refers to risk prediction of the severity of disease or of the probable course and clinical outcome associated with a disease. Thus, the term “method of prognosis” as used herein refers to methods by which the skilled person can estimate and/or determine a probability that a given outcome will occur.

The terms “stratification” or “stratifying” as used herein refer to the division of a population into subpopulations on the basis of specified criteria. More particularly, it refers to the division of a cohort of subjects into at least two groups on the basis of specific criteria, which in the context of the present invention comprise or consist of the results of the method of analysis.

### DETAILED DESCRIPTION

Provided herein are biomarkers associated with risk of bacterial infection, particularly hospital inquired infections, such as following surgeries. In particular, provided herein are fecal biomarkers (e.g., microbiomic, metabolomic, etc.) that correlate with elevated risk of infection in subjects following surgery (e.g., organ transplant surgery), and methods of treating and/or preventing such infections.

Commensal bacterial species produce metabolites that can activate systemic immune defenses and modulate inflammatory responses and a diverse microbiome and its metabolic products can stimulate the host immune system and support immune homeostasis. Production of bacterial metabolites, such as butyrate and secondary bile acids, can modulate development of inflammatory and regulatory T cell populations and provide defense against pathogens.

Experiments conducted during development of embodiments herein demonstrate that microbiome composition and a subset of microbiota-derived metabolites are independently associated, for example, with a subject's risk of post-surgical (e.g., liver transplant) infection.

The present disclosure provides a group of prognostic biomarkers (e.g., microbiomic biomarkers, metabolomic biomarkers, etc.) usable for assessing the relative risk of post-surgical infection. A method for assessing the risk of infection (e.g., hospital acquired, post-surgical, etc.) for a subject by measuring prognostic biomarkers (e.g., microbiomic biomarkers, metabolomic biomarkers, etc.) is also provided. For example, the method provided by the present disclosure is a non-invasive approach that utilizes body fluid or other sample (e.g., fecal samples) from a patient (e.g., hospitalized, pre-surgery, post-surgery, etc.) to determine the risk of bacterial infection.

Experiments conducted during development of embodiments herein identified prognostic biomarkers (e.g., microbiomic biomarkers, metabolomic biomarkers, etc.) that are present in different levels in subjects likely to suffer an infection (e.g., hospital acquired, post-surgery, etc.) and subject likely not to suffer such an infection. In some embodiments, analysis of such biomarkers in a sample (e.g., fecal sample) from a subject provides a prognosis for the likelihood of infection and indicates treatment courses of action that should be taken on behalf of the subject. In some embodiments, a panel of biomarkers is provided, wherein each individual biomarker is indicative of the infection, but together provide an enhanced determination as to the risk of infection for the subject. In some embodiments, methods are provided for assessing the levels of the various biomarkers herein (e.g., microbiomic biomarkers, metabolomic biomarkers, etc.). In some embodiments, the levels of one or more biomarkers herein are compared to controls and/or threshold values. In some embodiments, methods are provided for analyzing the levels of multiple biomarkers and providing a prognosis (e.g., low likelihood of infection, intermediate likelihood of infection, high likelihood of infection) and/or a treatment course of action based thereupon. In some embodiments, a score is provided based on the levels of one or more biomarkers. In some embodiments, the score indicates the risk of infection.

In some embodiments, provided herein are species or taxonomic groups of microbes (e.g., bacteria), the presence/absence or level of which is a subject or sample from the subject is prognostic of the relative likelihood of infection for a hospitalized subject or one undergoing surgery (e.g., liver transplant). In some embodiments, provided herein are panels of such microbiomic biomarkers.

In some embodiments, an increased level (e.g., above a threshold abundance) of one or more bacterial species is associated with increased risk of infection. In some embodiments, a decreased level (e.g., below a threshold abundance) of the one or more bacterial species is associated with a decreased risk of infection.

In some embodiments, a decreased level (e.g., below a threshold abundance) of one or more bacterial species is associated with increased risk of infection. In some embodiments, an increased level (e.g., above a threshold abundance) of the one or more bacterial species is associated with a decreased risk of infection.

In some embodiments, microbiomic biomarkers are assessed (e.g., quantified) is a sample form a subject by any suitable method. In some embodiments, a sample is tested to determine the microbiomic makeup of the sample. In some embodiments, specific species or taxonomic groups of bacteria are tested for (e.g., species of taxa that are prognostic of risk of infection). In some embodiments, intact bacteria are detected (e.g., by detecting surface polypeptides or markers). In other embodiments, bacteria are lysed and nucleic acids or proteins (e.g., corresponding to genes specific to the species of bacteria) are detected. In some embodiments, bacteria are identified using detection reagents (e.g., a probe, a microarray, e.g., an amplification primer) that specifically interact with a nucleic acid that identifies a particular species or taxa of bacteria.

Some embodiments, the methods provided herein comprise use of nucleic acid sequencing to detect, quantify, and/or identify gut microbiota. The term “sequencing,” as used herein, refers to a method by which the identity of at least 10 consecutive nucleotides (e.g., the identity of at least 20, at least 50, at least 100, or at least 200 or more consecutive nucleotides) of a polynucleotide are obtained. The term “next-generation sequencing” refers to the so-called parallelized sequencing-by-synthesis, sequencing-by-ligation platforms, nanopore sequencing methods, or electronic-detection based methods that will be understood in the field.

Some embodiments, the methods provided herein comprise acquiring a gut microbiota sample from a subject. As used herein, “gut microbiota sample” refers to a biological sample comprising a plurality of heterogeneous nucleic acids produced by a subject's gut microbiota. Fecal samples are commonly used in the art to sample gut microbiota. Methods for obtaining a fecal sample from a subject are known in the art and include, but are not limited to, rectal swab and stool collection. Suitable fecal samples may be freshly obtained or may have been stored under appropriate temperatures and conditions known in the art. Methods for extracting nucleic acids from a fecal sample are also well known in the art. The extracted nucleic acids may or may not be amplified prior to being used as an input for profiling the relative abundances of bacterial taxa, depending upon the type and sensitivity of the downstream method. When amplification is desired, nucleic acids may be amplified via polymerase chain reaction (PCR). Methods for performing PCR are well known in the art. Selection of nucleic acids or regions of nucleic acids to amplify are discussed above. The nucleic acids comprising the nucleic acid sample may also be fluorescently or chemically labeled, fragmented, or otherwise modified prior to sequencing or hybridization to an array as is routinely performed in the art.

In some embodiments, nucleic acids are amplified using primers that are compatible with use in, e.g., Illumina's reversible terminator method, Roche's pyrosequencing method (454), Life Technologies's sequencing by ligation (the SOLiD platform) or Life Technologies's Ion Torrent platform. Examples of such methods are described in the following references: Margulies et al (Nature 2005 437: 376-80); Ronaghi et al (Analytical Biochemistry 1996 242: 84-9); Shendure et al (Science 2005 309: 1728-32); Imelfort et al (Brief Bioinform. 2009 10:609-18); Fox et al (Methods Mol Biol. 2009; 553:79-108); Appleby et al (Methods Mol Biol. 2009; 513: 19-39) and Morozova et al (Genomics. 2008 92:255-64), which are incorporated by reference for the general descriptions of the methods and the particular steps of the methods, including all starting products, reagents, and final products for each of the steps.

In another embodiment, the isolated microbial DNA may be sequenced using nanopore sequencing (e.g., as described in Soni et al. Clin Chem 2007 53: 1996-2001, or as described by Oxford Nanopore Technologies). Nanopore sequencing technology is disclosed in U.S. Pat. Nos. 5,795,782, 6,015,714, 6,627,067, 7,238,485 and 7,258,838 and U.S. Patent Publication Nos. 2006/003171 and 2009/0029477.

The isolated microbial fragments may be sequenced directly or, in some embodiments, the isolated microbial fragments may be amplified (e.g., by PCR) to produce amplification products that sequenced. In certain embodiments, amplification products may contain sequences that are compatible with use in, e.g., Illumina's reversible terminator method, Roche's pyrosequencing method (454), Life Technologies' sequencing by ligation (the SOLiD platform) or Life Technologies' Ion Torrent platform, as described above. Embodiments herein are not limited by the techniques used to identify and/or quantify the bacteria present in a sample.

In some embodiments, provided herein are species or taxonomic groups of microbes (e.g., bacteria), the presence/absence or level of which is a subject or sample from the subject is prognostic of the relative likelihood of infection (e.g., hospital acquired, after surgery, etc.). In some embodiments, provided herein are panels of such microbiomic biomarkers.

Experiments conducted during development of embodiments herein demonstrate that the levels of various small molecule metabolites present in biological samples (e.g., stool sample, rectal swab, etc.) from a subject correlate with and are prognostic of the risk of infection for the subject. In some embodiments, provided herein are panels of metabolites, the levels of which in biological samples (e.g., stool sample, rectal swab, etc.) from a subject (e.g., hospitalized, pre-surgery, post-surgery, etc.) correlate with the rate of infection. In some embodiments, methods are provided of assessing the levels of such metabolomic biomarkers in a biological samples (e.g., stool sample, rectal swab, etc.) from a subject. In some embodiments, the metabolomic biomarkers in the panels herein and/or assessed herein include one or more biomarkers selected from indole-3-carboxaldehyde, indole-3-acetate, indole-3-propionate, desaminotyrosine, tryptamine, itaconate, crotonate, 3-aminoisobutyrate, 4-methylvalerate, omega-muricholic acid, gamma-muricholic acid, vanillin, synephrine, catechol, dopamine, tyrosine, etc.

In some embodiments, the levels of one or more of indole-3 indole-3-carboxaldehyde, indole-3-acetate, indole-3-propionate, desaminotyrosine, tryptamine, itaconate, crotonate, 3-aminoisobutyrate, 4-methylvalerate, omega-muricholic acid, gamma-muricholic acid, vanillin, synephrine, catechol, dopamine, tyrosine, etc. in a sample are quantified.

Any technique and/or instrumentation suitable for detecting/quantifying small molecules in a complex environment may find use in embodiments herein. In some embodiments, analytical platforms (e.g., High-throughput platforms, automated platforms, etc.) utilizing nuclear magnetic resonance (NMR) spectroscopy, gas chromatography (GC), liquid chromatography (LC), and/or mass spectrometry (MS) are employed to measure the metabolites within a biological sample. In some embodiments, NMR, GC, and/or LC coupled to MS is utilized.

Mass spectrometry can accurately identify/quantify thousands of metabolites within complex biological samples. In some embodiments, metabolites are detected/quantified in a biological sample using MS techniques, such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, tandem mass spectrometry (e.g., MS/MS, MS/MS/MS, ESI-MS/MS etc.), secondary ion mass spectrometry (SIMS), or ion mobility spectrometry (e.g. GC-IMS, IMS-MS, LC-IMS, LC-IMS-MS etc.). Mass spectrometry methods are well known in the art and have been used to quantify and/or identify biomolecules, such metabolites.

In certain embodiments, a gas phase ion spectrophotometer is used. In other embodiments, laser-desorption/ionization mass spectrometry is used to identify metabolites. Modern laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the metabolite is mixed with a solution containing a matrix, and a drop of the liquid is placed on the surface of a substrate. The matrix solution then co-crystallizes with the biomarkers. The substrate is inserted into the mass spectrometer. Laser energy is directed to the substrate surface where it desorbs and ionizes the proteins without significantly fragmenting them. However, MALDI has limitations as an analytical tool. It does not provide means for fractionating the biological fluid, and the matrix material can interfere with detection, especially for low molecular weight analytes. In SELDI, the substrate surface is modified so that it is an active participant in the desorption process. In one variant, the surface is derivatized with adsorbent and/or capture reagents that selectively bind the biomarker of interest. In another variant, the surface is derivatized with energy absorbing molecules that are not desorbed when struck with the laser. In another variant, the surface is derivatized with molecules that bind the biomarker of interest and that contain a photolytic bond that is broken upon application of the laser. In each of these methods, the derivatizing agent generally is localized to a specific location on the substrate surface where the sample is applied. The two methods can be combined by, for example, using a SELDI affinity surface to capture an analyte (e.g. biomarker) and adding matrix-containing liquid to the captured analyte to provide the energy absorbing material.

For additional information regarding mass spectrometers, see, e.g., Principles of Instrumental Analysis, 3rd edition., Skoog, Saunders College Publishing, Philadelphia, 1985; and Kirk-Othmer Encyclopedia of Chemical Technology, 4.sup.th ed. Vol. 15 (John Wiley & Sons, New York 1995), pp. 1071-1094; incorporated by reference in their entireties.

In some embodiments, the data from mass spectrometry is represented as a mass chromatogram. A “mass chromatogram” is a representation of mass spectrometry data as a chromatogram. Typically, the x-axis represents time and the y-axis represents signal intensity. In one aspect the mass chromatogram is a total ion current (TIC) chromatogram. In another aspect, the mass chromatogram is a base peak chromatogram. In other embodiments, the mass chromatogram is a selected ion monitoring (SIM) chromatogram. In yet another embodiment, the mass chromatogram is a selected reaction monitoring (SRM) chromatogram. In one embodiment, the mass chromatogram is an extracted ion chromatogram (EIC). In an EIC, a single feature is monitored throughout the entire run. The total intensity or base peak intensity within a mass tolerance window around a particular analyte's mass-to-charge ratio is plotted at every point in the analysis. The size of the mass tolerance window typically depends on the mass accuracy and mass resolution of the instrument collecting the data. As used herein, the term “feature” refers to a single small metabolite, or a fragment of a metabolite. In some embodiments, the term feature may also include noise upon further investigation.

In some embodiments, detection of the presence of a metabolite involves detection of signal intensity. This, in turn, can reflect the quantity and character of a biomarker. For example, in certain embodiments, the signal strength of peak values from spectra of a first sample and a second sample can be compared (e.g., visually, by computer analysis etc.) to determine the relative amounts of particular metabolites. Software programs such as the Biomarker Wizard program (Ciphergen Biosystems, Inc., Fremont, Calif.) can be used to aid in analyzing mass spectra. The mass spectrometers and their techniques are well known.

A person skilled in the art understands that any of the components of a mass spectrometer, e.g., desorption source, mass analyzer, detect, etc., and varied sample preparations can be combined with other suitable components or preparations described herein, or to those known in the art. For example, in some embodiments a control sample may contain heavy atoms, e.g. ¹³C, thereby permitting the test sample to be mixed with the known control sample in the same mass spectrometry run.

In some embodiments, a laser desorption time-of-flight (TOF) mass spectrometer is used. In laser desorption mass spectrometry, a substrate with a bound marker is introduced into an inlet system. The marker is desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of molecules of specific mass to charge ratio. In one embodiment of the invention, levels of metabolites are detected by MALDI-TOF mass spectrometry.

Methods of detecting metabolites also include the use of surface plasmon resonance (SPR). The SPR biosensing technology has been combined with MALDI-TOF mass spectrometry for the desorption and identification of metabolites.

Data for statistical analysis can be extracted from chromatograms (spectra of mass signals) using software for statistical methods known in the art. “Statistics” is the science of making effective use of numerical data relating to groups of individuals or experiments. Methods for statistical analysis are well-known in the art. In one embodiment a computer is used for statistical analysis. In one embodiment, the Agilent MassProfiler or MassProfilerProfessional software is used for statistical analysis. In another embodiment, the Agilent MassHunter software Qual software is used for statistical analysis. In other embodiments, alternative statistical analysis methods can be used. Such other statistical methods include the Analysis of Variance (ANOVA) test, Chi-square test, Correlation test, Factor analysis test, Mann-Whitney U test, Mean square weighted derivation (MSWD), Pearson product-moment correlation coefficient, Regression analysis, Spearman's rank correlation coefficient, Student's T test, Welch's T-test, Tukey's test, and Time series analysis.

In various embodiments, signals from mass spectrometry are transformed in different ways to improve the performance of the method. Either individual signals or summaries of the distributions of signals (such as mean, median or variance) can be so transformed. Possible transformations include taking the logarithm, taking some positive or negative power, for example the square root or inverse, or taking the arcsin (Myers, Classical and Modern Regression with Applications, 2nd edition, Duxbury Press, 1990).

In some embodiments, the ability to quantitate the amount of a metabolite (or multiple metabolite) in a biological sample from a subject allows a clinician to make assessments regarding the condition of the subject, to further provide a diagnosis or prognosis for the subject, and/or to further recommend or administer a treatment course of action. Thus, according to another aspect of the present invention there is provided a method of characterizing the risk of infection comprising determining the abundance of at least one metabolite in a biological sample from the subject (e.g., stool sample, rectal swab, etc.), comparing the quantity to a control or threshold value, and providing a prognosis regarding the likelihood of infection and/or a treatment course of action for the subject.

Once the level of one or more metabolites is measured by the techniques described herein and/or understood in the field, it is typically compared to a level of that metabolite in a control subject or a threshold value. In some embodiments, if the metabolite level is above or below the control or threshold, determinations about the state or the subject can be inferred or concluded. In embodiments in which the levels of multiple metabolites are measured to provide a prognosis or to determine a treatment course of action, an algorithm may be employed to combine the level of the multiple biomarkers, and/or their levels relative to individual thresholds, into a single prognosis or treatment course of action. In some embodiments, a score is provided for each metabolite, based on the abundance of the metabolite in the sample relative to a control or threshold value. In some embodiments, the combination of scores from multiple metabolites is used to provide a prognosis and/or determine a treatment course of action. In some embodiments, if a metabolite level is above a threshold it is given a first score (e.g., 1 or 0) and if the metabolite level is below a threshold it is given a second score (e.g., 0 or 1). In some embodiments, a metabolite level is scored based on how far above or below the metabolite level is relative to a threshold (e.g., scored 0-100). In some embodiments, the scores of multiple metabolites are combined to provide a prognosis (e.g., likelihood of infection (e.g., qualitative (e.g., high, intermediate, or low), percentage (e.g., 50%, 60%, 70%, 80%, 90%, etc.), etc.)). In some embodiments, the combined score of multiple metabolites (e.g., based on comparison to thresholds) is compared to a ‘panel threshold’ or multiple panel thresholds to classify the subject and the likelihood of infection. In some embodiments, comparison to a panel threshold or multiple panel thresholds allows for stratification (e.g., qualitative or quantitative) of the likelihood of infection. In some embodiments, examples of a qualitative likelihood of infection are low risk, intermediate risk, high risk, severe risk, etc. In some embodiments, examples of a quantitative likelihood of infection are 10%, 20%, 30%, 40%, 50%, 60%, 70%, 80%, 90%, 95%, etc. Individual and/or combined metabolite scores, based on comparison to controls or threshold values, can be determined and combined in any suitable manner to achieve a statistically relevant prognosis. The scope herein is not limited by the various ways of comparing metabolite levels to thresholds and combining them to produce a prognosis and/or treatment course of action.

In some embodiments, a biological sample is obtained from a subject. In some embodiments, the biological sample is tested for biomarkers (e.g., microbiomic, metabolomic, etc.). In some embodiments, a biological sample is a fecal sample. The fecal sample can be collected from feces after defecation, such as a stool sample, or from feces obtained directly from the rectum of a subject. In some embodiments, a biological sample is gastrointestinal lavage fluid (GLF). In some embodiments, the biological sample is tested directly for biomarkers (e.g., microbiomic, metabolomic, etc.). In some embodiments, the biological sample is treated prior to analysis. For example, a sample may be fractionated, centrifuged, liquefied, diluted, etc. prior to analysis. In some embodiments, the biological sample is obtained from a subject within 30 days of a surgery. For example, in some embodiments the biological sample is obtained from the subject no more than 30 days before a surgery, or no more than 30 days after a surgery. In some embodiments, the biological sample is obtained from the subject within 30 days, within 20 days, within 10 days, or within 5 days of a surgery. In some embodiments, an analysis is performed to provide a clinician with a prognosis and treatment course of action.

In some embodiments, the methods herein provide decision-making information regarding a treatment course of action. In some embodiments, methods herein comprise a step of administering a treatment of prophylactic for an infection.

In some embodiments, methods herein identify subjects are risk (e.g., low, moderate, high, severe, etc.) of suffering an infection. In some embodiments, subjects identified as being at risk of infection (e.g., moderate risk, high risk, or severe risk of infection (e.g., following surgery) are administered treatment including but not limited to antibiotics, metabolite supplementation, microflora replacement therapy, etc. In some embodiments, high or severe risk are administered microbiome augmentation. Examples of microbiome augmentation include fecal augmentation therapy, antibiotics, prebiotics, probiotics (i.e., live biotherapeutic products), metabolite supplementation, microflora replacement therapy, or microbiota-derived metabolites. In some embodiments, patients identified to be at risk are isolated. For example, patients identified to be at risk (e.g., high or severe risk patients) may be isolated to limit contact with other people, who may otherwise expose the high or severe risk patient to parasites. In some embodiments, patients identified to be at risk are provided further surgical procedures.

### EXPERIMENTAL

### Example 1

To determine the dynamic between the microbiota, microbiota-derived metabolites, MDRO expansion in the gut and invasive infection in liver transplant recipients, a prospective surveillance study assessing fecal microbial composition and microbe-derived metabolite concentrations was performed. Results demonstrated that expansion of MDRO populations in the fecal microbiota correlates with increased risks of infection and that patients with low concentrations fecal SCFA, secondary bile acids and a subset of indole compounds are more likely to have Enterococcus expansion. Further, specific microbiome derived metabolite profiles identified patients with lost diversity as well as postoperative bacterial infection.

### Results:

### Microbiome Compositions in Liver Transplant Patients is Widely Variable

A prospective fecal microbiome and metabolome study was conducted on patients admitted to the University of Chicago Medical center for liver transplantation. 158 patients were enrolled, of whom 28 did not undergo transplantation and 23 did not provide a fecal sample from 7 days prior to 30 days after transplantation (FIG. 7). For the 107 patients who underwent LT and from whom fecal samples were collected, the causes of end-stage liver disease were varied and included alcoholic cirrhosis/hepatitis (56%), malignancy (21%), non-alcoholic fatty liver disease (15%), and others (Table 1).

Fecal microbiota compositions in LT recipients was determined by shotgun metagenomic sequencing in the peri-transplant period. MetaPhlAn-4 was implemented to determine the relative composition of microbial communities (Blanco-Miguez A et al. Extending and improving metagenomic taxonomic profiling with uncharacterized species with MetaPhlAn 4). Microbiome composition and diversity varied widely between the 107 patients, with some approximating healthy donors (HD) and others being dominated by single bacterial taxa (FIG. 1). To determine whether microbiome compositions influence post-transplant outcomes, LT patients were divided into three groups on the basis of microbiota diversity, with 27 high diversity patients falling within the range of diversities detected in the HD group and the remaining 80 LT patients evenly divided into low and medium diversity groups. A taxUMAP of the 107 LT patients and 21 healthy donors revealed close clustering between the subset of high diversity LT and HD fecal samples while most of LT patient samples harbored microbiota with distinct and wide-ranging compositions (FIG. 1).

While high diversity LT patient and HD microbiome compositions shared some similarities, LT patients had significantly lower relative abundance of Oscillospiraceae (synonym Ruminococcaceae) (W(48)=117, p<0.001, two-tailed test), and higher abundance of Enterobacterales (W(48)=487, p<0.001. two-tailed). In contrast, low and medium diversity LT patients differed from high diversity patients in nearly every measured metric. In addition to reduced diversity, the low and medium diversity groups had lower abundances of the phylum Bacteroidetes (W(64)=57, p<0.001, two-tailed; W(67)=342, p=0.014, two-tailed, respectively) and families Lachnospiraceae (W(64)=0, p<0.001, two-tailed; W(67)=140, p<0.001, two-tailed, respectively) and Oscillospiraceae (W(64)=32, p<0.001, two-tailed; W(67)=190, p<0.001, two-tailed, respectively). Increases in Enterococcus and Enterobacterales (p<0.001) abundance were common in low and medium diversity LT patients. Many increases amounted to >90% of the entire composition of the microbiome, with 40% of LT patients having greater than 20% Enterococcus relative abundance and 17% of patients having greater than 5% Enterobacterales relative abundance.

To ensure stool samples corresponded to the perioperative period, a histogram of stool sample collections was created (FIG. 8). The median day to stool collection was post-operative day 4, with most samples being collected within the first 7 days before or after transplant. There was no difference in stool collection timing between diversity groups.

### Microbiome Derived Metabolite Abundances Differ Among LT Recipients.

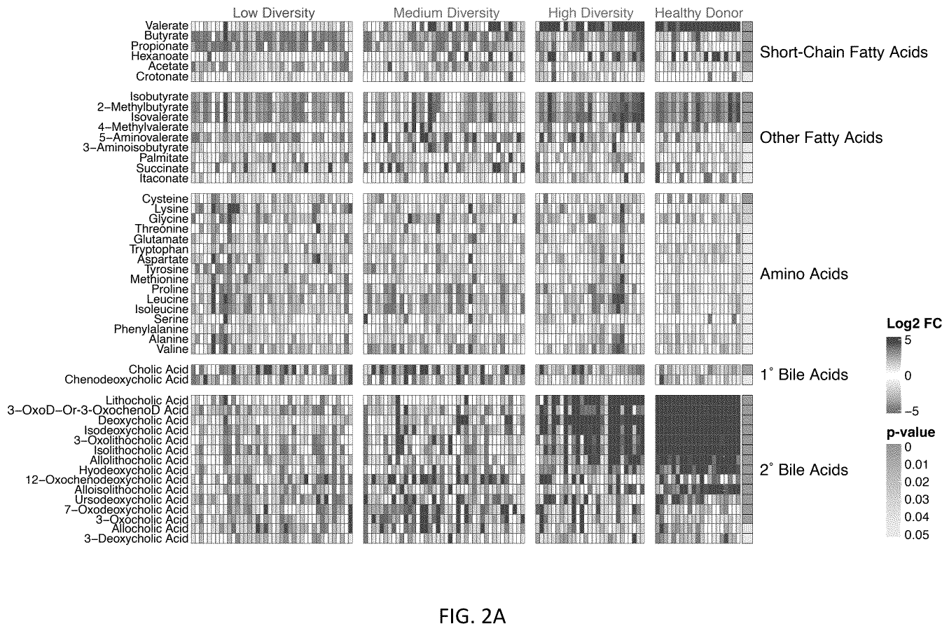

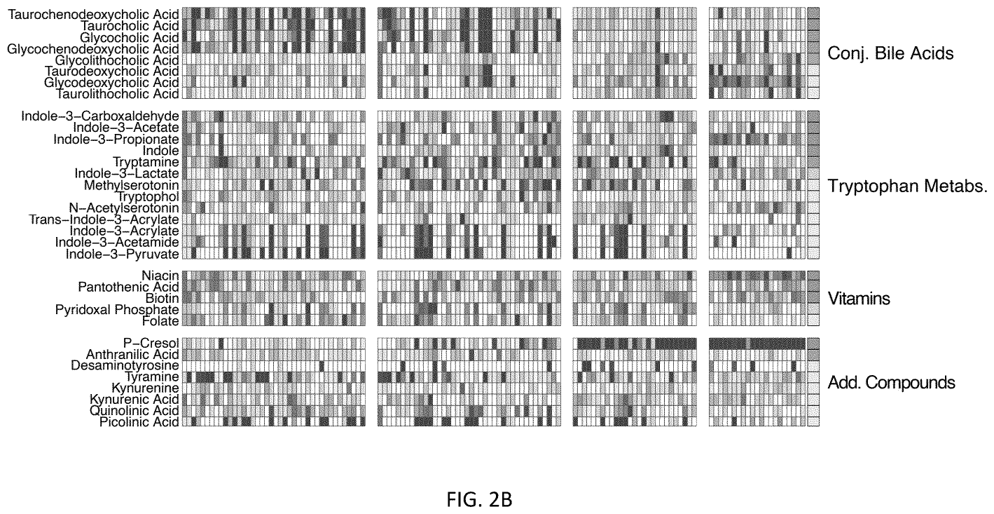

Targeted GC- and LC-MS analyses was performed for metabolites produced or modified by the gut microbiota on peri-transplant fecal samples from the LT patient cohort (FIG. 2). Within the LT patient cohorts, relative amounts of butyrate, valerate, and hexanoate varied dramatically, with patients in the low and medium diversity cohorts having markedly reduced levels while those in the high diversity group approached the range seen in the healthy donors.

Markedly reduced concentrations of secondary bile acids and corresponding increases in concentrations of conjugated and primary bile acids were detected among low and medium diversity LT patients, indicating loss of microbial bile acid deconjugating and 7a-dehydroxylation capacity. Fecal samples from LT recipients had increased abundance of amino acids, however this increase did not correlate with microbiota diversity or composition and thus may be a consequence of chronic liver disease and/or recent liver transplantation.

Microbiota derived vitamins biotin, pantothenic acid, niacin, and folate were reduced in low diversity LT patients, likely reflecting loss of gut commensal bacteria that produce B vitamins, in particular species belonging to the phylum Bacteroidetes. P-Cresol, a microbiome derived phenol linked to cardiovascular risk in chronic kidney disease, was also reduced in low diversity LT patients. Only one microbiome derived metabolite, tyramine, which is produced by Enterococcus faecalis and Enterococcus faecium, is significantly more abundant in liver transplant recipients.

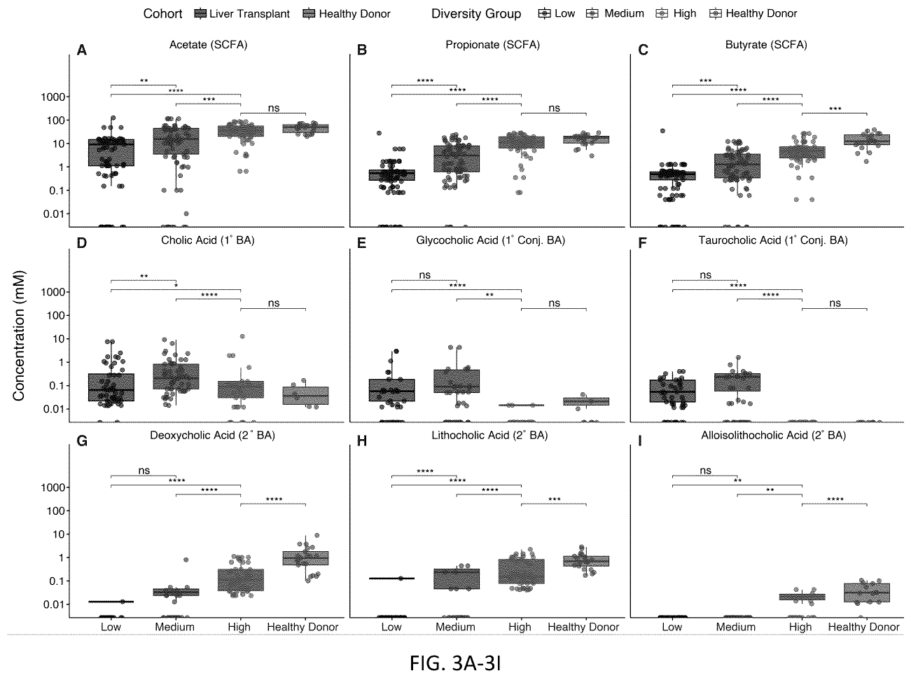

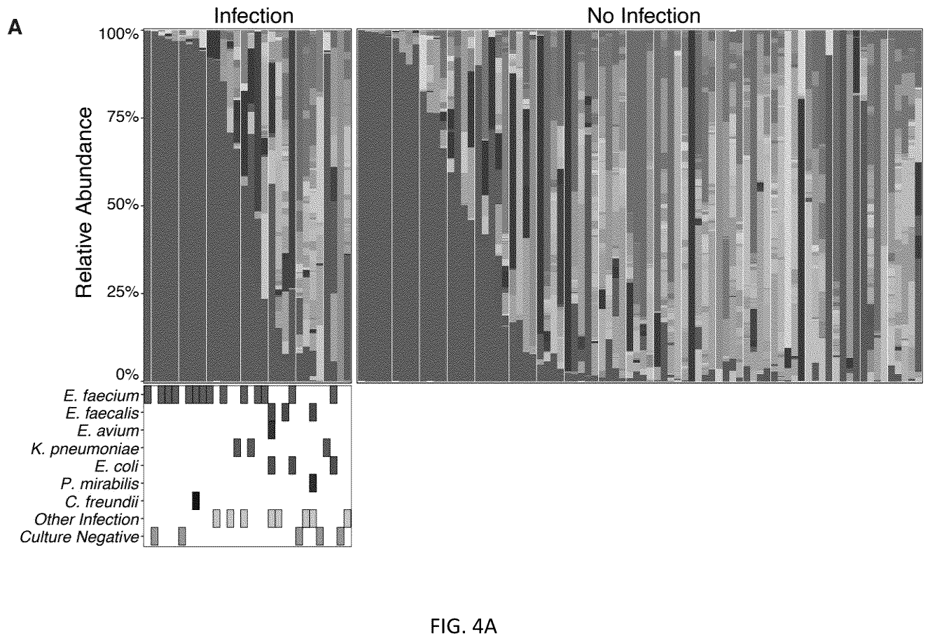

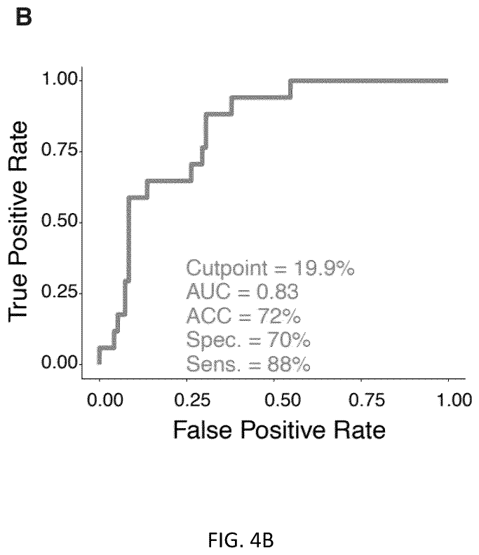

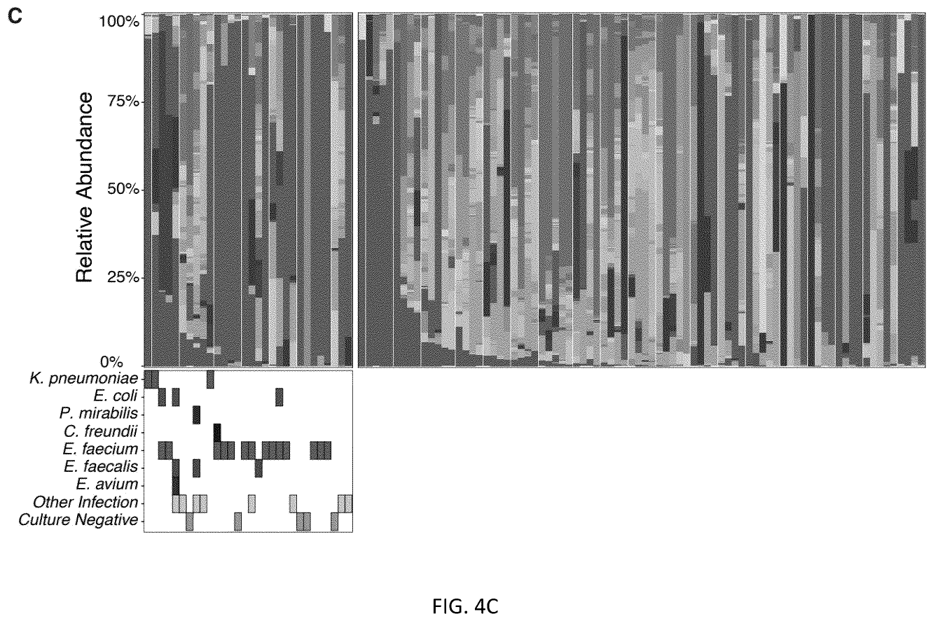

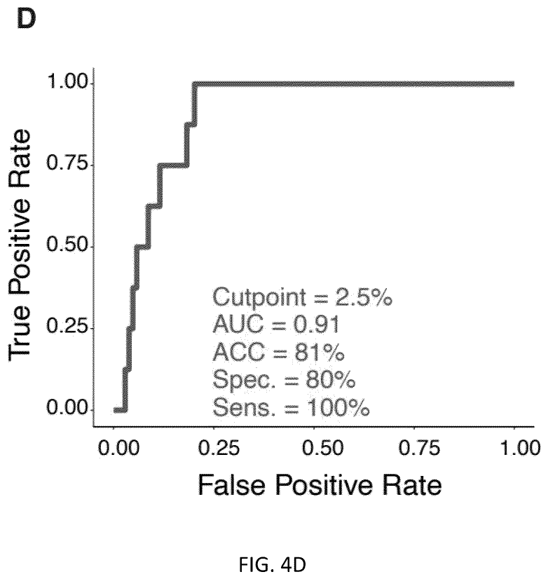

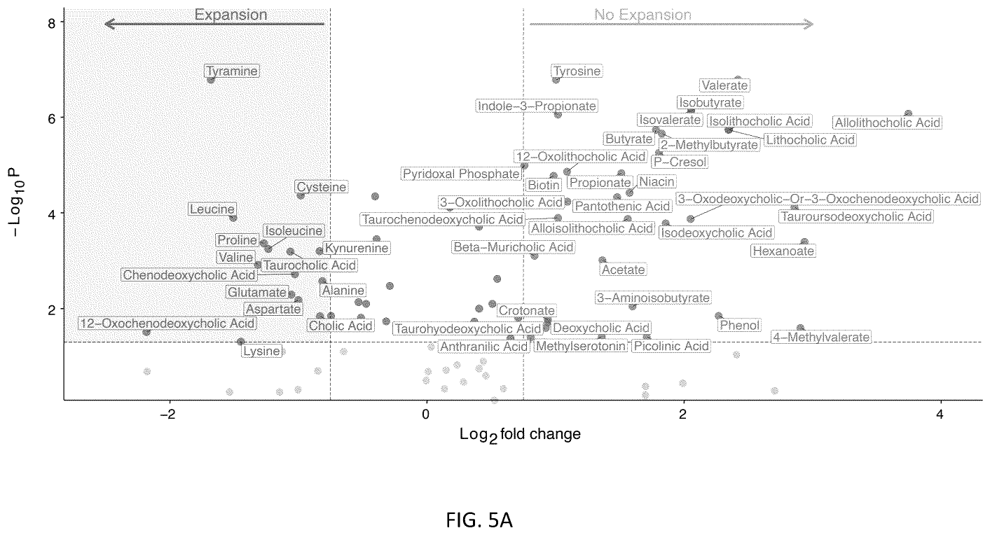

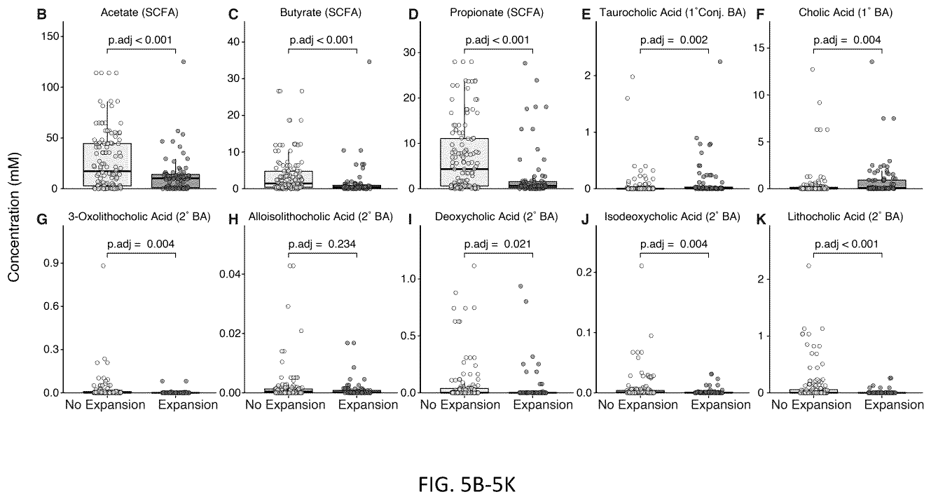

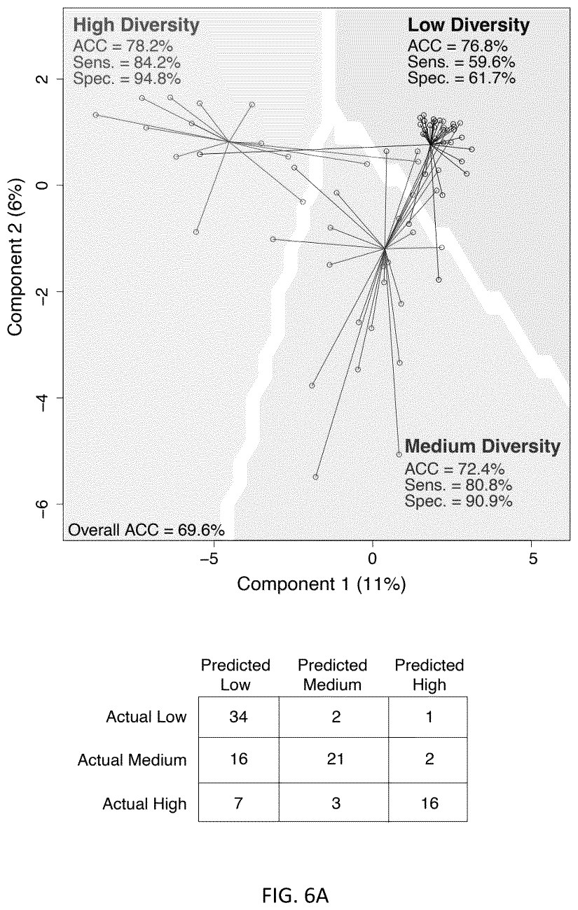

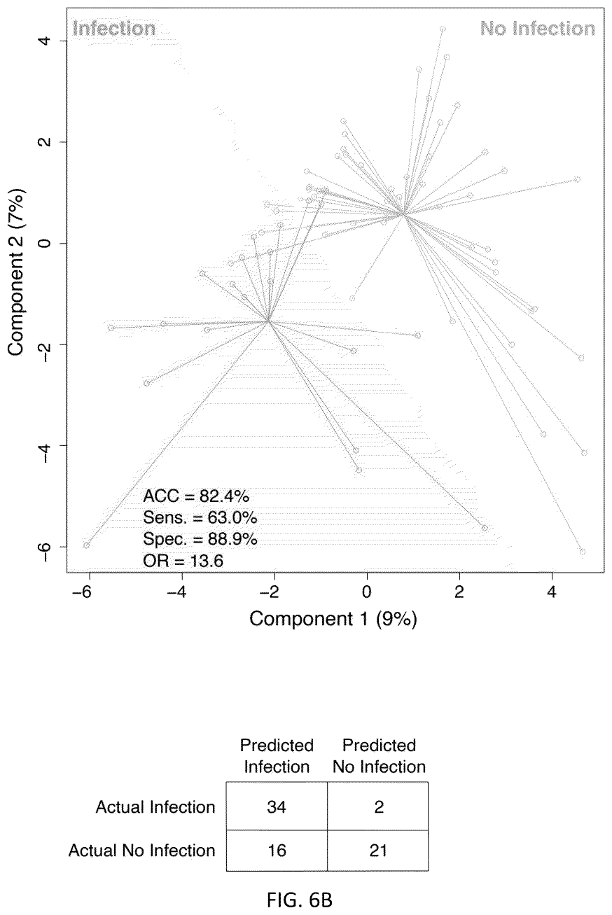

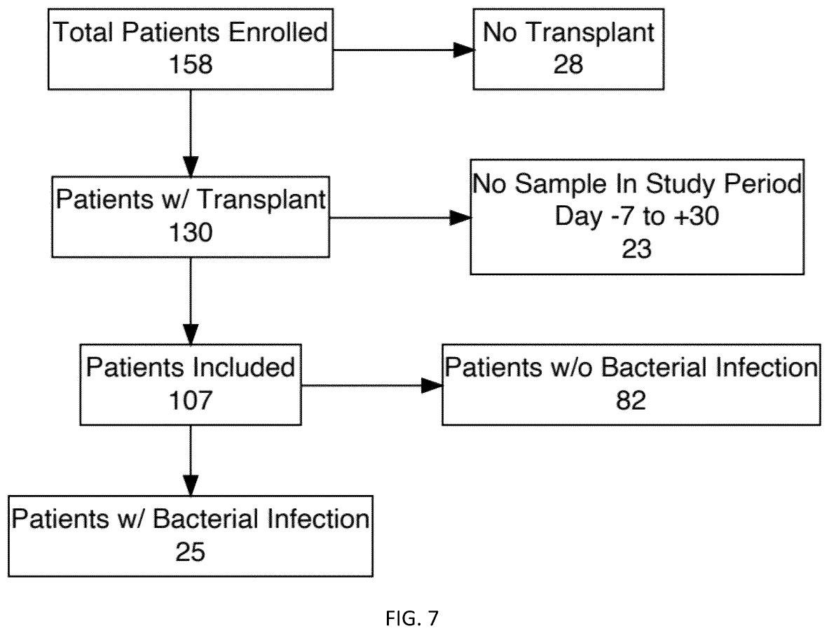

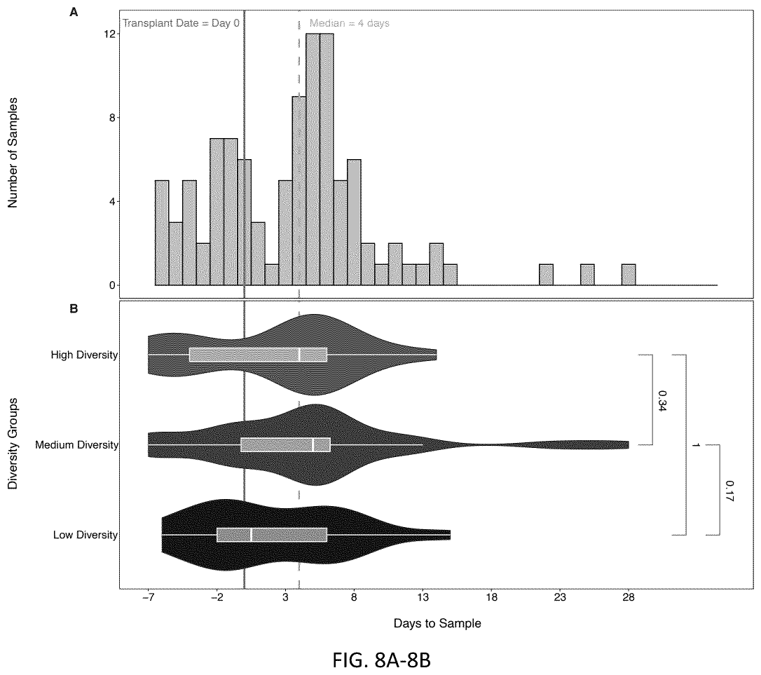

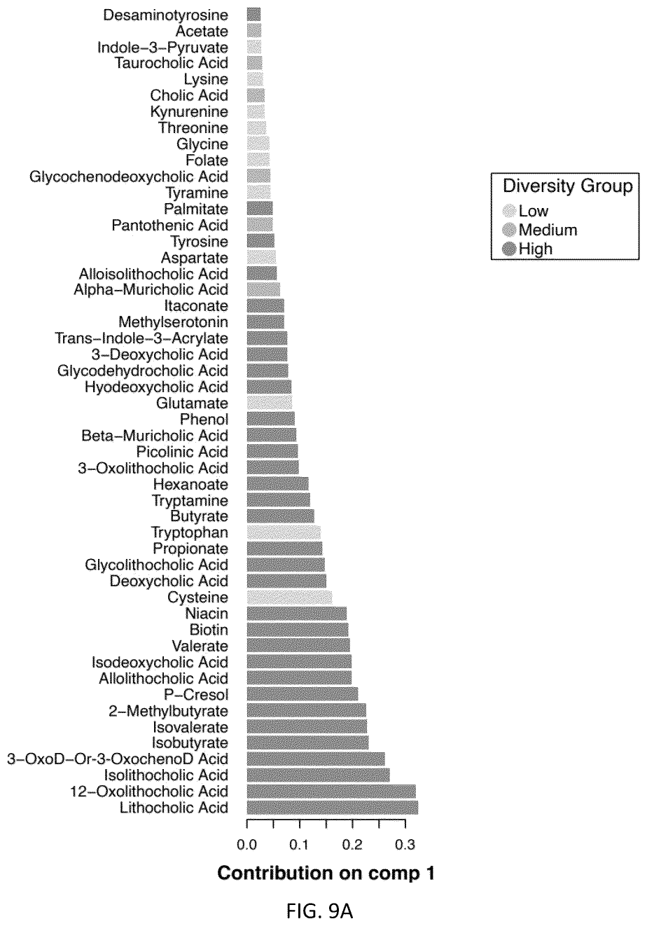

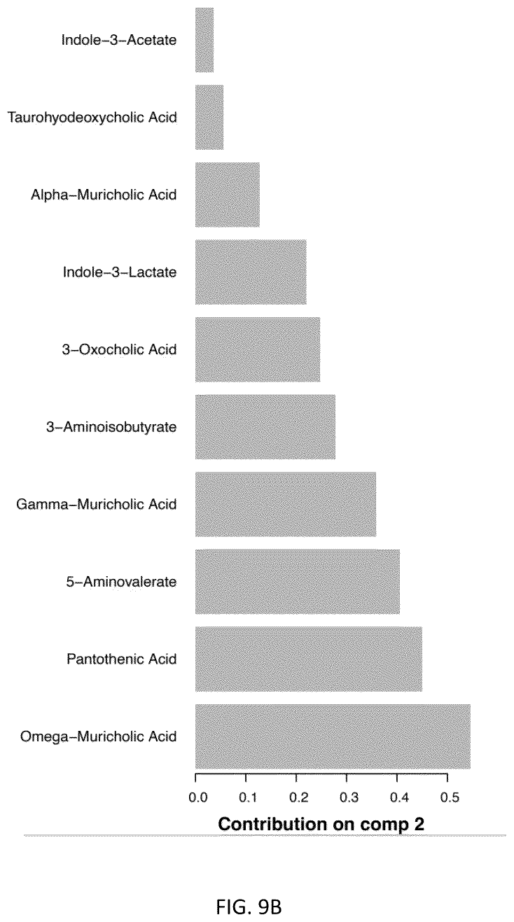

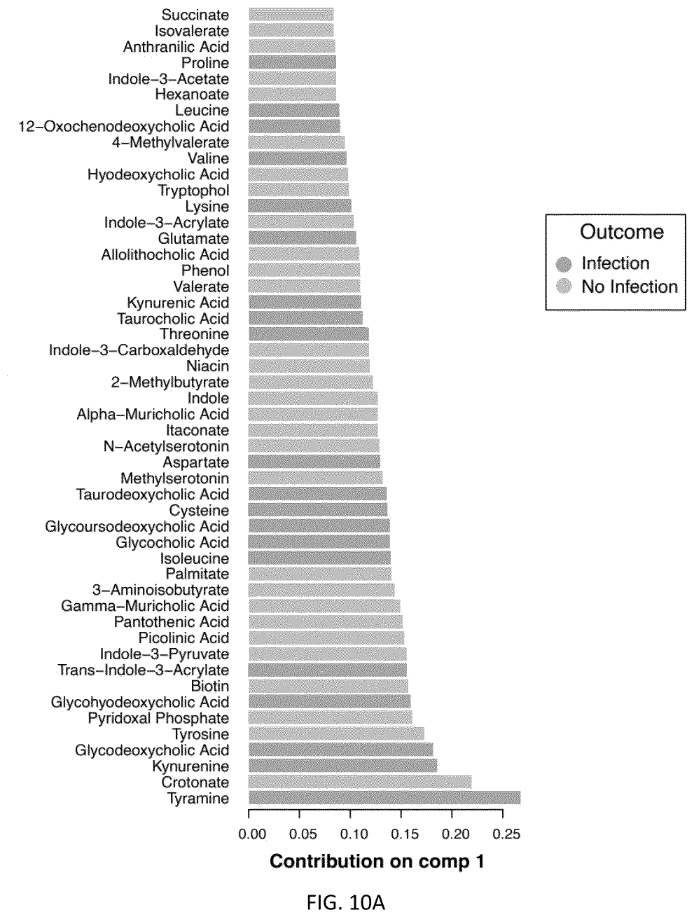

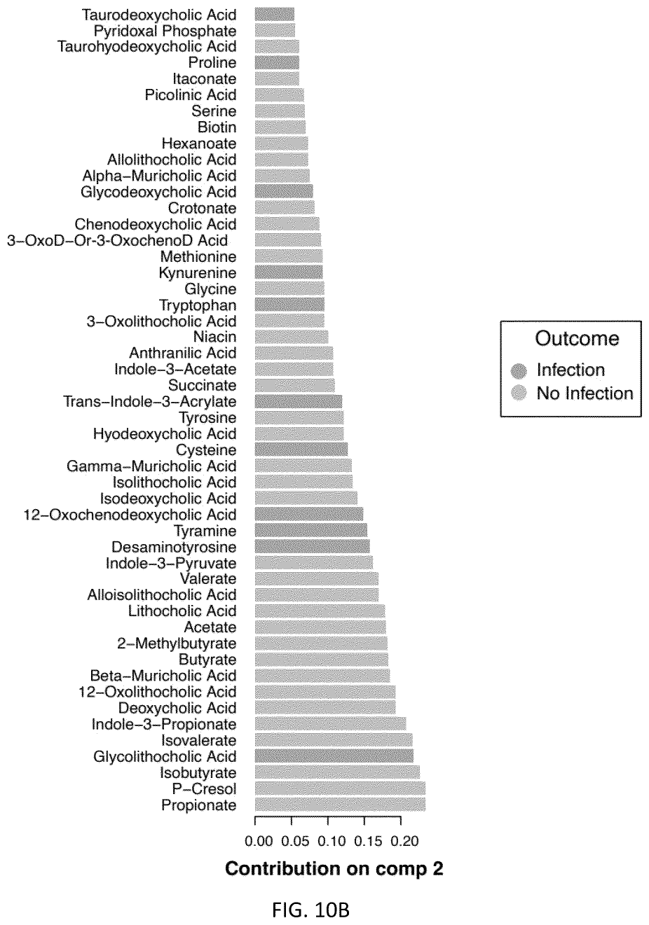

Mass spectrometric quantitation of acetate, propionate and butyrate concentrations in fecal samples from LT patients revealed marked reductions in patients with low microbiota diversity (FIG. 3) while the high diversity LT patients had SCFA concentrations in the same range as fecal samples from healthy subjects. Conjugated and unconjugated primary bile acids were present at very low concentrations in fecal samples from healthy subjects and high diversity LT patients while several low and medium diversity fecal samples had much higher concentrations. In contrast, fecal secondary bile acid concentrations were markedly lower in low and medium diversity compared to high diversity samples. Quantitation of fecal metabolites reveals the remarkable range of metabolic activity in the lower intestinal tract microbiota of LT patients. A subset of patients with near normal microbiota diversity having metabolite concentrations within the normal range while the majority of LT patients, in particular those with the lowest microbiota diversities, were shown to have markedly reduced and in many cases absolute absence of beneficial metabolites.

Clinical, Taxonomic, and Metabolomic Characteristics Associated with Postoperative Infection

To determine whether taxonomic changes in microbiome composition or changes in microbial metabolism impact the risk for postoperative infection, LT patients were evaluated for infections in the first 30 days following transplant. Of the 107 LT recipients, 25 developed bacterial infection (FIG. 7). The taxonomic composition of stool samples collected closest to the time of infection were compared to peri-transplant samples from patients who did not develop infection. There were no significant differences in the age, sex, or race between patients with or without infection (Table 1). Additionally, there were no differences in indication for transplant, Model of End Stage Liver Disease-Na (MELD-Na) score, use of mechanical ventilation, renal replacement therapy, vasopressors, immunosuppression, or antibiotic administration between the groups. The most common site of infection was intra-abdominal (50%), followed by urinary tract (20%), and skin (13%) (Table 2). The most common organisms causing infection were members of the genus Enterococcus (45%) and order Enterobacterales (20%). No specific organism was identified in 5 (13%) infections.

TABLE 1
Demographic and clinical characteristics of LT patients with and without infection.

| | No Infection | | Infection | | p | | No Infection | | Infection | | p |
|---|---|---|---|---|---|---|---|---|---|---|---|
| Demographics # (%) | | | | | | Disease Severity # (%) | | | | | |
| Male | 44 | (53.7) | 16 | (64.0) | 0.88 | MELD-Na med [IQR] | 28 | [19, 33] | 30 | [19, 33] | 0.5 |
| Age med [IQR] | 55 | [41, 63] | 59 | [46, 68] | 0.35 | Dialysis | 22 | (27.2) | 9 | (34.6) | 1 |
| Race | | | | | .88 | Pressers | 6 | (7.4) | 5 | (19.2) | 1 |
| White | 54 | (65.4) | 17 | (65.4) | | Ventilation | 5 | (6.2) | 2 | (7.7) | 1 |
| Black/African-American | 9 | (1.1) | 2 | (7.7) | | Immunosuppression | | | | | |
| More than one Race | 8 | (9.9) | 2 | (7.7) | | Basiliximab | 54 | (66) | 18 | (72) | 1 |
| Asian/Mideast Indian | 6 | (7.4) | 2 | (7.7) | | Tacrolimus | 82 | (100) | 25 | (100) | 1 |
| AI or AN | 1 | (1.2) | 0 | (0.0) | | Mycophenolate | 72 | (89) | 24 | (96) | 1 |
| Declined | 4 | (4.9) | 1 | (3.8) | | Corticosteroid | 82 | (100) | 25 | (100) | 1 |
| Unknown | 0 | (0.0) | 2 | (7.7) | | Antibiotics | | | | | |
| Indication | | | | | | Pip/Tazo | 70 | (85) | 23 | (92) | 1 |
| Alcoholic Cirrhosis | 40 | (48.8) | 8 | (32) | 0.88 | Cefepime | 26 | (32) | 11 | (44) | 1 |
| Malignancy | 17 | (21.0) | 6 | (23.1) | 1 | Ceftriaxone | 16 | (20) | 7 | (28) | 1 |
| NAFLD/NASH | 10 | (12.3) | 6 | (23.1) | 0.88 | Meropenem | 4 | (4.9) | 1 | (4.0) | 1 |
| Alcoholic Hepatitis | 6 | (7.4) | 2 | (7.7) | 1 | Ciprofloxacin | 15 | (18) | 4 | (16) | 1 |
| PSC | 6 | (7.4) | 0 | (0.0) | 0.88 | Vancomycin IV | 42 | (51) | 17 | (68) | 1 |
| Autoimmune | 5 | (6.2) | 0 | (0.0) | 0.88 | Vancomycin PO | 1 | (1.2) | 1 | (4.0) | 1 |
| Cryptogenic | 4 | (4.9) | 1 | (3.8) | 1 | Metronidazole | 34 | (42) | 12 | (48) | 1 |
| Acute Viral Hepatitis | 4 | (4.9) | 0 | (0.0) | 0.88 | Rifaximin | 37 | (45) | 12 | (48) | 1 |
| Chronic Hepatitis C | 4 | (4.9) | 0 | (0.0) | 0.88 | Gentamicin | 1 | (1.2) | 1 | (4.0) | 1 |
| Wilson's Disease | 2 | (2.5) | 1 | (3.8) | 1 | Tobramycin | 4 | (4.9) | 3 | (12) | 1 |
| Chronic Hepatitis B | 0 | (0.0) | 1 | (3.8) | 0.88 | Daptomycin | 1 | (1.2) | 3 | (12) | 0.83 |
| Hemochromatosis | 0 | (0.0) | 1 | (3.8) | 0.88 | Total | 82 | | 25 | | |
| DILI | 1 | (1.2) | 0 | (0.0) | 1 | | | | | | |
| Other | 8 | (9.8) | 5 | (20.0) | 0.88 | | | | | | |

Variables are represented in Table 1 as number and percent or median and interquartile range. Other indication for transplant includes: congestive hepatopathy, primary biliary cirrhosis, and unidentified. Immunosuppressive medications were included if given within the 30 day post operative period. Antibiotics were included if given in the first 7 pre-operative days or the day of LT. p-values obtained by Chi-squared goodness of fit and adjusted for multiple comparisons. Abbreviations: AI-American Indian; AN-Alaska Native; NAFLD-Non-Alcoholic Fatty Liver Disease; NASH-Non-Alcoholic Steatohepatitis; PSC-Primary Sclerosing Cholangitis; DILI-Drug Induced Liver Injury; MELD-Na—Model for End State Liver Disease Na; Pip/Tazo—Piperacillin/Tazobactam; IV-Intravenous; PO—per orem.

Expansion of Enterococcus and Enterobacterales is Associated with Postoperative Infection

Given that Enterococcus and Enterobacterales were the most common causes of infection, it was next investigated whether increases of these taxa was associated with the risk of invasive infection. Perioperative fecal samples were ordered by relative abundance of Enterococcus or Enterobacterales and compared to causes of infection. Infections caused by Enterococcus clustered with fecal samples with increased densities of Enterococcus, while Enterobacterales infections occurred in patients with increased fecal Enterobacterales density (FIG. 4). In some instances, patients with expansion of pathobiont species did not develop invasive infections, suggesting that other unidentified factors, such as immune and mucosal barrier defenses and other clinical factors likely also contribute to the risk of infection.

Receiver operator characteristic (ROC) analyses were performed to determine correlations between intestinal expansion of pathobionts with infection and optimized for sensitivity and specificity. The optimized threshold determined for Enterococcus relative abundances predicting Enterococcus infections was 19.9%. At this threshold expansion of Enterococcus predicts infection with sensitivity of 88% ([CI]: 65%-100%), specificity of 70% ([CI]: 59%-94%) and an area under the curve of 0.83 ([CI]: 0.74-0.91) (FIG. 4). Expansion of Enterococcus >19.9% occurred in 41% of LT patients and accounts for 15 of the 17 (88%) of Enterococcus infections (Table 3). For Enterobacterales, a threshold of 2.5%, relative abundance predicted infection with sensitivity of 100% ([CI]: 100%-100%), specificity of 80% ([CI]: 72%-94%) and an area under the curve of 0.91 ([CI]: 0.82-0.96) (FIG. 4). Enterobacterales expansion at or above 2.5% occurred in 27% of LT patients and accounted for all 8 (100%) Enterobacterales infections.

TABLE 3
Enterococcus and Enterobacterales expansion in
the gut microbiome predicts postoperative infection.

| A | ≥19.9% Rel. | <19.9% Rel. | |
|---|---|---|---|
| Enterococcus | Abd. | Abd. | Total |
| Infected | 15 | 2 | 17 |
| Uninfected | 29 | 66 | 95 |
| Sensitivity | 88.2% | | |
| Specificity | 69.5% | | |
| Odds Ratio | 17.1 | | |

| B | ≥2.5% Rel. | <2.5 Rel. | |
|---|---|---|---|
| Enterobacterales | Abd. | Abd. | Total |
| Infected | 8 | 0 | 8 |
| Uninfected | 21 | 83 | 104 |
| Sensitivity | 100% | | |
| Specificity | 79.8% | | |
| Odds Ratio | Inf | | |

As shown in Table 3, part A, 44/112 (41%) of included patients had Enterococcus expansion ≥19.9% or higher. 88% (15/17) of infectious caused by Enterococcus were associated with expansion of 19.9% or greater. Odds ratio for Enterococcus infection was 17.1. As shown in Table 3, part B, 29/112 (27%) of included patients had Enterobacterales expansion of 2.5% or higher and all 100% (8/8) of infectious caused by Enterobacterales were associated with expansion of 2.5% or more. The Odds ratio for Enterobacterales approached infinity, as no infections occurred in those <2.5%. In combination, only 2/25 (8%) of Enterococcus or Enterobacterales infections occurred without any expansion.

Microbiome Derived Metabolites Associated with Enterococcus Expansion

To determine whether fecal metabolite concentrations identify patients with Enterococcus or Enterobacterales expansion, volcano plots were prepared comparing log₂ fold changes of metabolites (relative to the median value of each compound) against statistical significance, with p-values adjusted for multiple comparisons following the Benjamini-Hochberg method (FIG. 5). Expansion was defined as >=19.9% for Enterococcus and 2.5% for Enterobacterales, as determined in FIG. 4. A variety of metabolites were enriched in samples without Enterococcus expansion and included several secondary bile acids: lithocholic, isolithocholic, allolithocholic, isodeoxycholic, alloisolithocholic, 3-oxolithocholic, and 12-oxolithocholic acids. Butyrate, valerate, propionate, and hexanoate were also enriched in patients without Enterococcus expansion. Other microbially derived metabolites that were enriched in patients without expansion included the tryptophan-derived metabolite indole-3-propionate, the vitamin biotin, and a number of branched chain fatty acids. Fifteen metabolites were enriched in patients with Enterococcus expansion, including multiple conjugated bile acids, likely indicating reduced microbiome-mediated bile acid deconjugation. Tyramine, a tyrosine derived amine produced by E. faecium and E. faecalis and several amino acids were also enriched in expanded samples.

To confirm the trends established by qualitative analysis, a subset of metabolites with previously established beneficial effects were measured quantitatively against standardized controls. Of these, many of the same SCFA and secondary bile acids were enriched in samples without expansion. (FIG. 5) The primary bile acid cholic acid and conjugated bile acid taurocholic acid were enriched in samples with expansion. The smaller number of LT patients with fecal Enterobacterales expansion precluded identification of statistically significant metabolite associations after correcting for multiple comparisons.

### Metabolomic Model Identifies Diversity Groups and Post-Operative Infection

Given the speed of metabolomic profiling compared to metagenomic sequence analysis and the interest in real-time identification of patients for potential microbiota reconstitution therapies, it was next investigated how accurately fecal metabolite concentrations predict microbiota diversity and the risk of infection. Using 93 metabolite measurements per fecal sample, sparse partial least squares discriminant analysis (sPLS-DA) (le Cao K-A et al., mixOmics: Omics Data and Integration Project (2016); CRAN.R-project.org/package=mixOmics) predicted low, medium, and high diversity groups with high sensitivity, specificity, and accuracy (FIG. 6A) 70% ([CI]: 59.7%-78.3%). Secondary bile acids, short- and branched-chain fatty acids correlated most significantly with high diversity microbiota (FIG. 9). The model most accurately identified low diversity fecal samples, only misidentifying 1 of 37 as high diversity. Exogenous factors such as diet may also influence the metabolic activity of resident commensal bacteria.

Fecal metabolite levels also accurately predict pathobiont dissemination beyond the gut to sites of postoperative infection such as the peritoneal cavity, urinary tract, or bloodstream (FIG. 6B). Samples obtained from patients with infection clustered distinctly from those without infection, with an overall accuracy of 82% ([CI]: 73.9%-89.1%), a strong odds ratio (13.6 ([CI]: 4.8-38.6) and high specificity of 89% ([CI]: 80%-94.8%). Sensitivity for infection was slightly lower 63% ([CI]: 42.4%-80.6%), likely reflecting multifactorial causality of infection following LT. To determine which metabolites made the greatest contribution to the PLS-DA models, metabolite loadings for each axis were assembled in separate plots (Supplemental FIG. 4). The largest contributors to the model's first two components were tyramine, conjugated bile acids, indole derivatives, and SCFAs in patients who remained uninfected.

### DISCUSSION

LT patients follow a distinct path from health to end-stage liver disease (ESLD). Some patients develop acute, fulminant hepatitis over the course of days, while others progressively lose hepatic function over many years due to chronic, progressive fibrosis leading to cirrhosis and end stage complications. The wide range of microbiome compositions detected in LT cohorts likely result from diversity of diseases leading to ESLD, differing treatments modalities, including exposure to antibiotics for infections such as spontaneous bacterial peritonitis and acquisition of antibiotic-resistant pathobionts. Herein it is demonstrated that targeted mass spectrometric analysis of fecal samples identifies LT patients with partial to complete loss of metabolites that impact pathobiont fitness, host immune defenses, and mucosal barrier integrity. Loss of beneficial metabolites closely correlates with loss of bacterial taxa that constitute a diverse microbiota and identifies patients with an increased risk of postoperative infections caused by antibiotic-resistant pathobionts. The findings herein also indicate that a diverse microbiome, with the capacity to produce SCFAs, secondary bile acids and AHR ligands, enhances resistance of LT patients to pathobiont expansion in the gut lumen and invasive infection from the gut.

The contributions of commensal bacterial species and their metabolic products to disease resistance in clinical settings are challenging to disentangle. Treatment with broad-spectrum antibiotics, the most common cause of microbiome compromise, was common in the cohort herein, which results in concurrent depletion of many microbial taxa and microbiota-derived metabolites. Loss of beneficial Bacteroidetes, Lachnospiraceae and Oscillospiraceae are paralleled by loss of SCFAs, secondary bile acids and indole compounds. It is likely that disease resistance results from the aggregate contributions of different components. The results herein indicate that therapeutic interventions to re-establish missing commensal bacterial populations and normalize metabolite profiles can treat or prevent bacterial infection following surgery, such as following liver transplant.

Metagenomic analyses of fecal samples require deep nucleic sequencing platforms and bioinformatic analyses that continue to evolve as bacterial genome databases grow, microbial nomenclature changes, and microbial gene annotation improves. Targeted metabolomic analyses by mass spectrometry, on the other hand, are rapid, accurate, and bioinformatic platforms to define features and provide chemical structures are well established. The findings herein demonstrate that patients with compromised microbiomes, as defined by metagenomic analyses, can be identified by measuring fecal SCFAs, bile acids, and a narrow range of microbially generated metabolites. Not only can patients with reduced diversity be identified, but also those at increased risk for invasive bacterial infection. Given the rapidity with which microbiome compositions can change from one day to another, obtaining same-day results would provide critical, real-time insights to guide clinical interventions to improve microbiome compositions and functions, such as therapy with live biotherapeutic products and/or microbiome modulating foods.

In conclusion, targeted fecal metabolite measurements are shown herein to identify a large subset of patients undergoing LT with markedly deficient microbiome compositions. The ability to rapidly identify patients with an increased risk of postoperative infection can be used to select patients for treatment to preserve or reconstitute microbiome functions and prevention of exposure to antibiotic-resistant pathobionts.

### Methods:

### Patient Enrollment:

Patients eligible for LT or having recently undergone LT at University of Chicago Medicine were recruited to the protocol (IRB20-0163). Healthy subjects were recruited from the University of Chicago campus and were screened for absence of recent antibiotic treatment and autoimmune or other chronic illnesses prior to fecal microbiome and metabolome characterization.

### Definition of Postoperative Infection:

All patient records were screened for documentation of clinical infection during the study period. Screening was performed by author CL at 1-month intervals. Infection was defined as any positive test for infection (microbiological, molecular, biochemical, or radiographic) that was documented as a true positive by the treating clinical team in the medical record. The majority of infections were microbiologically defined by culture or polymerase chain reaction. Infections were classified by location, microbiology, and antimicrobial resistance patterns (Table 2). Infections were included for comparison if the infection occurred within the first 30 postoperative days and a stool sample was collected within 14 days prior or 2 days following the development of the infection. Each infection was paired with a stool sample collected nearest to the diagnosed infection. In patients who did not develop infection, the stool sample closest to the day of liver transplant was used for comparison.

TABLE 2
Characteristics of infections.

| | #(%) | |
|---|---|---|
| Enterococcus | 18 | (45) |
| E. faecium | 14 | (35) |
| E. faecalis | 3 | (7.5) |
| E. avium | 1 | (2.5) |
| Enterobacterales | 8 | (20) |
| E. coli | 3 | (7.5) |
| K. pneumoniae | 3 | (7.5) |
| P. mirabilis | 1 | (2.5) |
| C. freundii | 1 | (2.5) |
| Other Bacteria | 14 | (35) |
| S. aureus | 3 | (7.5) |
| P. aeruginosa | 2 | (5) |
| S. maltophilia | 1 | (2.5) |
| Bacteroides sp. | 1 | (2.5) |
| C. difficile | 1 | (2.5) |
| H. pylori | 1 | (2.5) |
| Culture Negative | 5 | (12.5) |
| Site of Infection | | |
| Abdominal | 15 | (50) |
| Skin/Surg. Site | 5 | (16.7) |
| Urinary | 6 | (20) |
| Blood Stream | 2 | (6.7) |
| Lower Airway | 2 | (6.7) |

As shown in Table 2, Enterococci accounted for most infection, 45%. Followed by Enterobacterales 20%. The remaining 35% of infection were caused by various other bacteria. The total number of organisms identified is 40, which is greater than the number of infections (30) because of polymicrobial infections. Abbreviations: Surg-Surgical.

### Inclusion/Exclusion:

Patients were included in this analysis if they received a LT and had a stool sample collected within peri-operative period: days −7 to +30. Patients were excluded from analysis if they developed an infection, but a stool sample was not collected within 14 days preceding or 2 days following the diagnosis of the infection. 158 patients were enrolled in the study. 28 patients had not received a LT, 23 patients did not have a stool sample collected within postoperative day −7 to +30, and all patients had a stool sample collected within −14 to +2 days relative to the diagnosis of an infection (FIG. 7). The remaining 107 patients were included for analysis. 25 patients developed a bacterial infection and 82 did not. 40 unique infections occurred in the cohort because some patients developed more than one infection.

### Specimen Collection:

After enrollment, stool samples were collected before and after LT. All stool samples were collected while admitted at University of Chicago Medical Center. Collection was started immediately after study enrollment and continued for up to two years. Each sample was immediately refrigerated and frozen within 24 hours of collection. Microbiota compositions and metabolite profiles were then characterized.

### Specimen Analysis:

DNA Extraction: DNA was extracted using the QIAamp PowerFecal Pro DNA kit (Qiagen). Prior to extraction, samples were homogenized by mechanical disruption using a bead beater. Briefly, samples were suspended in a bead tube (supplied by Qiagen) with lysis buffer and homogenized on a bead mill (Fisherbrand). Samples were then centrifuged, and supernatant was resuspended in a reagent that effectively removed inhibitors. DNA was then purified routinely using a spin column filter membrane and quantified using Qubit 2.0 fluorometer.

Shotgun Sequencing: Libraries were prepared using 100-200 ng of genomic DNA using the QIAseq FX DNA library kit (Qiagen). Briefly, DNA was fragmented enzymatically into smaller fragments and desired insert size was achieved by adjusting fragmentation conditions. Fragmented DNA was end repaired and ‘A's’ were added to the 3′ends to stage inserts for ligation. During ligation step, Illumina compatible Unique Dual Index (UDI) adapters were added to the inserts and the prepared library was amplified. Libraries were then cleaned up, and library size was measured using a TapeStation (Agilent). Sequencing was performed on the Illumina NextSeq 500 platform producing 5-10 million reads. Adapters were trimmed off from the raw reads, and their quality were assessed and controlled using Trimmomatic (v.0.39), (43) then human genome was removed by kneaddata (v0.7.10, https://github.com/biobakery/kneaddata). Taxonomy was profiled using metaphlan4 using the resultant high-quality reads. Alpha diversity of fecal samples was calculated using the Inverse Simpson and Shannon indices. Taxonomic uniform manifold approximate projections (taxUMAP) were used to visualize beta diversity (https://github.com/jsevo/taxumap).

Metabolomic analysis: SCFAs were derivatized with pentafluorobenzyl bromide (PFBBr) and relatively quantified via negative ion collision induced-gas chromatography-mass spectrometry. Four SCFA's were quantified absolutely: acetate, butyrate, succinate, and propionate ([-]CI-GC-MS, Agilent 8890). Eight bile acids (primary: cholic acid; conjugated primary: glycocholic acid, taurocholic acid; secondary: deoxycholic acid, lithocholic acid, isodeoxycholic acid, alloisolithocholic acid, and 3-oxolithocholic acid) were quantified (m/mL) by negative mode liquid chromatography-electrospray ionization-quadrupole time-of-flight-MS ([-]LC-ESI-QTOF-MS, Agilent 6546). Eleven indole metabolites were quantified by UPLC-QqQ LC-MS. Seventy additional compounds were relatively quantified using normalized peak areas relative to internal standards.

### Statistical Analysis:

All analysis was conducted using R statistical language (v4.2.2 (2022-10-31)) using the RStudio (v2022.12.0+353) integrated development environment. Non-parametric, descriptive statistics, were used to describe the clinical characteristics of patients. All clinical tables were generated using the R package gtsummary (Sjoberg D D, et al., Reproducible Summary Tables with the gtsummary Package. R J 2021; 13(1):570). For categorical clinical variables, a Chi-square goodness-of-fit test was implemented. All other continuous variables regressed against categorical variables (e.g. relative abundance vs diversity groups or age vs infection group) were statistically analyzed using a two-tailed, Wilcoxon-rank sum test, from the R package rstatix (Kassambra A. rstatix: Pipe-Friendly Framework for Basic Statistical Tests_. R package version 0.7.1 (2022) cran.r-project.org/package=rstatix) unless otherwise noted. All p-values were adjusted to account for multiple comparisons following the Benjamini-Hochberg method. Adjusted p-values of the tests were considered to be statistically significant for all analyses conducted if p ≤0.05.

To compare relatively quantified metabolites, log₂ fold-change (log₂ FC) was calculated per compound using the median value across all fecal samples. These log₂ FC values were then arranged by statistical significance when comparing the diversity groups (Kruskal-wallis test) and visualized on a heatmap using the ComplexHeatmap package (https://github.com/jokergoo/ComplexHeatmap). Volcano plots, visualized via the R package Enhanced Volcano (https://github.com/kevinblighe/EnhancedVolcano), used the same log₂ FC method as mentioned, but were statistically compared using two-tailed, Wilcoxon-rank sum tests. Receiver operator characteristic curves (ROC) were generated using the R package cutpointr (Thiele C, Hirschfeld G. Cutpointr: Improved estimation and validation of optimal cutpoints in r. J Stat Softw 2021; 98) with the predicting variable as relative abundance of either Enterococcus or Enterobacterales and the outcome variable as Enterococcus or Enterobacterales infection, respectively. ROC curves were optimized for the Youden index and visualized using ggplot2 (Wickham H. ggplot2. New York, NY: Springer New York; 2009). Sparse partial least squares discriminatory analysis (sPLS-DA) was constructed using the R package mixOmics and required an input matrix of samples by metabolites. Models were tuned using 5-fold cross-validation repeated 50 times (mixOmics::perf and mixOmics::tune.splsda). The main hyperparameters that required tuning were the number of optimal components to retain in the model, number of metabolites to keep in the final model, as well as distance metric, for which we used the maximum distance “max.dist”. The final model was built (mixOmics::splsda) and was used to predict classes of either diversity group or infection (predict). Background predictions (shaded areas on FIG. 6), were constructed using the final sPLS-DA model (mixOmics::background.predict). Additional model metrics for predicting bacterial infection were obtained via the R package caret::confusionMatrix (Kuhn M. Caret: classification and regression training. Astrophysics Source Code Library 2015; asc1:1505.003) or epiR::epi.tests (Stevenson M et al. epiR: Tools for the Analysis of Epidemiological Data. R package version 2.0.53 (2022) CRAN.R-project.org/package=epiR.).

Since the model for predicting diversity groups included a multi-class prediction, additional model metrics were obtained from using the R package mltest::ml_test (Dudnik G. mltest: Classification Evaluation Metrics. R package version 1.0.1 (2018) cran.r-project.org/package=mltest). The inclusion flow diagram was built using the R package PRISMAstatement::flow_exclusions (RISMAstatement: Plot Flow Charts According to the “PRISMA” Statement. R package version 1.1.1 (2019) CRAN.R-project.org/package=PRISMAstatement). All data and code are available on both Github (https://github.com/DFI-Bioinformatics/Microbiome_Liver_Transplant) and Zenodo (https://zenodo.org/badge/latestdoi/592530758).

1. A method of assessing risk of infection in a subject, the method comprising assessing the level of one or more microbiomic biomarkers and/or one or more metabolomic biomarkers in a fecal sample obtained from the subject.

2. The method of claim 1, wherein the one or more microbiomic biomarkers comprise a relative abundance of Enterococcus species and/or a relative abundance of Enterobacterales species in the fecal sample.

3. The method of claim 2, wherein the subject is indicated to be at risk of having or currently experiencing an infection when:
a) the relative abundance of Enterococcus species in the fecal sample is greater than or equal to a threshold value for Enterococcus species; and/or
b) the relative abundance of Enterobacterales species in the fecal sample is greater than or equal to a threshold value for Enterobacterales species.

4. The method of claim 3, wherein the threshold value for Enterococcus species is 19.9% and/or the threshold value for Enterobacterales species is 2.5%.

5. The method of claim 1, wherein the one or more metabolomic biomarkers are selected from tyramine, kynurenine, acetate, butyrate, propionate, taurocholic acid, cholic acid, 3-oxolithocholic acid, chenodeoxycholic acid, 12-oxochenodeoxycholic acid, deoxycholic acid, isodeoxycholic acid, and lithocholic acid.

6. The method of claim 5, wherein increased levels of one or more of tyramine, kynurenine, taurocholic acid, cholic acid, chenodeoxycholic acid, or 12-oxochenodeoxycholic acid and/or decreased expression of one or more of acetate, butyrate, propionate, 3-oxolithocholic acid, deoxycholic acid, isodeoxycholic acid, lithocolic acid, tyrosine, valerate, hexanotate, indole-3-propionate, allolithocholic acid, isolithocholic acid, alloisolithocholic acid, crotonate, isobutyrate, or isovalerate in the fecal sample indicates that the subject is at risk of having or currently experiencing an infection.

7. The method of claim 1, further comprising providing microbiome augmentation to the subject when the subject is determined to be at risk of having or currently experiencing an infection.

8. The method of claim 1, wherein the subject is preparing to undergo a surgery or has received a surgery.

9. The method of claim 8, wherein surgery is an organ transplant.

10. The method of claim 9, wherein the organ transplant is a liver transplant.

11. The method of claim 1, wherein the fecal sample is obtained from the subject within 30 days of the surgery.

12. A method comprising assessing the level of one or more microbiomic biomarkers and/or one or more metabolomic biomarkers in a fecal sample obtained from a subject within 30 days of a surgery on the subject.

13. The method of claim 12, wherein the one or more microbiomic biomarkers comprise a relative abundance of Enterococcus species and/or a relative abundance of Enterobacterales species in the fecal sample.

14. The method of claim 13, wherein the subject is indicated to be at risk of having or currently experiencing an infection when:
a) the relative abundance of Enterococcus species in the fecal sample is greater than or equal to a threshold value for Enterococcus species; and/or
b) the relative abundance of Enterobacterales species in the fecal sample is greater than or equal to a threshold value for Enterobacterales species.

15. The method of claim 14, wherein the threshold value for Enterococcus species is 19.9% and/or the threshold value for Enterobacterales species is 2.5%.

16. The method of claim 12, wherein the one or more metabolomic biomarkers are selected from tyramine, kynurenine, acetate, butyrate, propionate, taurocholic acid, cholic acid, 3-oxolithocholic acid, chenodeoxycholic acid, 12-oxochenodeoxycholic acid, deoxycholic acid, isodeoxycholic acid, and lithocholic acid.

17. The method of claim 16, wherein increased levels of one or more of tyramine, kynurenine, taurocholic acid, cholic acid, chenodeoxycholic acid, or 12-oxochenodeoxycholic acid and/or decreased expression of one or more of acetate, butyrate, propionate, 3-oxolithocholic acid, deoxycholic acid, isodeoxycholic acid, lithocolic acid, tyrosine, valerate, hexanotate, indole-3-propionate, allolithocholic acid, isolithocholic acid, alloisolithocholic acid, crotonate, isobutyrate, or isovalerate in the fecal sample indicates that the subject is at risk of having or currently experiencing an infection.

18. The method of claim 12, further comprising providing microbiome augmentation to the subject when the subject is determined to be at risk of having or currently experiencing an infection.

19. The method of claim 12, wherein the surgery is an organ transplant.

20. The method of claim 19, wherein organ transplant is a liver transplant.
